(19) United States
(12) Patent Application Publication
(10) Pub. No.: US 2005/0202539 A1
(43) Pub. Date: Sep. 15, 2005

(54) System for screening eukaryotic membrane proteins

(75) Inventors: Jayhong A. Chong, Brookline, MA (US); Magdalene M. Moran, Brookline, MA (US); David E. Clapham, Wellesley, MA (US)

Correspondence Address: FISH & NEAVE IP GROUP;ROPES & GRAY LLP, ONE INTERNATIONAL PLACE, BOSTON, MA, 02110-2624, US

(73) Assignee: Hydra Biosciences, Cambridge, MA (US)

(21) Appl. No.: 11/078,188

(22) Filed: Mar. 11, 2005

Related U.S. Application Data
(60) Provisional application No. 60/552,175, filed on Mar. 11, 2004.

(51) Int. Cl. C12P 21/04; C12P 21/06; C12N 1/12

(52) U.S. Cl. 435/69.7; 435/252.1

(57) ABSTRACT

The present invention provides methods and compositions for expressing eukaryotic membrane proteins.

### RELATED APPLICATIONS

This application claims priority to U.S. provisional application 60/552,175, filed Mar. 11, 2004, the disclosure of which is hereby incorporated by reference in its entirety.

### BACKGROUND OF THE INVENTION

A variety of membrane proteins exist to mediate ion flux across cellular membranes. The proper expression and function of membrane proteins is essential for the maintenance of cell function, intracellular communication, and the like. Exemplary membrane proteins including ion channels, transporters, ionotropic receptors, and gap junctions are expressed throughout embryonic and adult development in every tissue and cell type. Accordingly, methods and compositions to facilitate the study of membrane proteins, as well as methods and compositions for identifying agents that can modulate the activity of one or more membrane protein has tremendous utility.

Ion channels are one class of membrane protein contemplated by the present invention. Ion channels are macromolecular protein tunnels that span the cell membrane's lipid bilayer. While approximately 30% of the energy expended in cells goes to maintain the ionic gradient across the cell membrane, it is the ion channel that spends this stored energy, much as a switch releases the electrical energy of a battery. Ion channels are efficient compared to enzymes; small conformational changes gate a single channel from closed to open to allow up to 107 ions to flow per second. A few picoamps of highly selected ions flow during the channel opening. Since they are efficient, the number of ion channels per cell is relatively low; a few thousand channels of a given subtype/cell are usually sufficient to perform their task while orders of magnitude higher numbers of receptors or enzymes are required to carry out their tasks. Ion channels are usually classified by the type of ion they selectively pass (sodium, potassium, calcium, or chloride) although some are indiscriminate. Different ion channels are activated (or gated) by either extracellular ligands, transmembrane voltage, or intracellular second messengers.

Voltage-gated potassium (Kᵥ), sodium (Naᵥ), calcium (Caᵥ) channels, and non-selective cation channels regulate the flow of ions into and out of cells and thereby support specialized higher order cell functions such as excitability, contraction, secretion, and synaptic transmission (B. Hille, Ion Channels of Excitable Membranes (Sinauer Associates, Sunderland, Mass., 3d. ed., 2001)). Hundreds of Kᵥ, Naᵥ, and Caᵥ channel proteins provide the tremendous functional diversity required for the complex behaviors of eukaryotic vertebrate and invertebrate cell-types (C. M. Armstrong, B. Hille, Neuron 20: 371-80 (1998); W. A. Catterall, Neuron 26: 13-25 (2000)). Ion channels are also widespread in prokaryotes but their gating and function are poorly understood because few have been functionally expressed in a system in which their properties can be studied.

Defective ion channel proteins are responsible for cystic fibrosis, long QT syndrome, heritable human hypertension (Liddle's syndrome), disorders in glucose homeostasis with familial persistent hyperinsulinemic hypoglycemia of infancy, hereditary nephrolithiasis (Dent's disease), and a variety of hereditary myopathies including generalized myotonia (Becker's disease), myotonia congenita (Thomsen's disease), periodic paralyses, malignant hyperthermia, and central core storage disease. The defects underlying cystic fibrosis all occur in a single gene encoding a chloride-permeant ion channel known as the cystic fibrosis transmembrane conductance regulator (CFTR). The elucidation of the mechanisms underlying these diseases will benefit medicine as a whole, not just those with a particular disease. For instance, although inherited long QT syndrome is not a routine diagnosis, dissecting its molecular basis, defects in potassium channels: HERG and KVLQT1, and sodium channels (SCN5A) will benefit the study of ventricular arrhythmias responsible for 300,000 sudden, natural deaths each year. Likewise, although a defect in the recently cloned epithelial sodium channel (ENaC) is the basis of a very rare form of inherited hypertension (Liddle's syndrome or pseudoaldosteronism), normal ENaC might serve as an alternative target in attempts to correct the physiological defects created by the mutant CFTR molecule, and work with it will provide insight into the mechanism of essential hypertension. Ligands and drugs that regulate ion channels may be useful in studying these disease mechanisms.

Ion channel blockers have been used to treat a number of cardiovascular diseases such as high blood pressure, angina, and atrial fibrillation. Atrial fibrillation alone affects over 2.5 million people in the United States, with an estimated 160,000 new cases diagnosed each year, and an estimated annual cost of $1 billion. Thus, ligands or drugs that modulate ion channel activity may be useful in treating these diseases.

The primary structural theme of ion-selective channels is of a pore region surrounded by two transmembrane (2TM) segments. The first high resolution images of a bacterial 2TM tetrameric channel revealed the structural basis of K⁺ ion selectivity encoded by the signature GY/FG sequence (D. A. Doyle et al., Science 280, 69-77 (1998)). In the primary structure of voltage-sensitive ion channels, an additional four transmembrane segments precede the pore-containing domain. The pore-forming subunits (α₁) of Naᵥ, and Caᵥ, are composed of 4 homologous repeats of 6TM domains (C. Sato et al., Nature 409, 1047-51 (2001)). In theory, gene duplication of the 6TM Kᵥ channels provided the precise structural requirements for highly selective Na⁺ and Ca²⁺ channels. In particular, selectivity for Ca²⁺ requires coordination of the Ca²⁺ ions by four negatively charged glutamic or aspartic acid residues lining the pore. The TRP class of ion channels are presumably tetramers of single 6TM subunits, but only a subset of these channels are moderately Ca²⁺ selective (D. E. Clapham, L. W. Runnels, C. Strubing, Nat. Rev. Neurosci. 2, 387-96 (2001)).

Gap junctions, ionotropic receptors, and ion transporters also play critical roles in cellular signaling and maintenance of the electrochemical gradient. Gap junctions play critical roles in cell-cell communication. Ionotropic receptors, otherwise known as ligand gated channels, are essential for proper neurotransmission, muscle contraction and blood pressure. Transporters serve multiple functions. These include but are not limited to maintaining the electrochemical gradient and regulating the intracellular concentration of rare ions like iron. Mutations in all three classes of proteins can cause disease in humans.

Studies of a number of membrane proteins have been hindered by difficulties expressing these proteins in certain in vitro systems. For example, a number of ion channels have not been successful candidates for screening for compounds that modulate their activity due to an inability to achieve functional expression of the channels in mammalian or other systems suitable for high throughput screening. Accordingly, there exists a need for improved methods and compositions suitable for facilitating screening for agents that modulate the activity of eukaryotic membrane proteins. Such membrane proteins (e.g., proteins that mediate membrane flux) include, but are not limited to ion channels, gap junctions, transporter proteins, and ionotropic receptors. Agents that modulate the activity of a membrane protein (e.g., a protein that mediates membrane flux) can be used to treat various disorders, defects, or injuries relating to misregulation or mutation of a membrane protein. Exemplary disorders, defects, and injuries include, but are not limited to, disorders of the central nervous system, peripheral nervous system, gastrointestinal tract, cardiovascular system, reproductive system (e.g., male or female reproductive system), renal system, hepatic system, lymphatic system, immune system, respiratory system, excretory system, endocrine system, and muscular skeletal system.

### BRIEF SUMMARY OF THE INVENTION

Accordingly, it is an object of the invention to provide methods and compositions for studying eukaryotic membrane proteins (e.g., proteins that mediate membrane flux), and in particular, for screening for agents that modulate the activities of such proteins. Provided are methods and compositions for expressing eukaryotic membrane proteins in prokaryotic cells, and using these prokaryotic cells for screening assays to identify agents that modulate the activity of the membrane protein. Such agents can be used to modulate the activity of the membrane protein, and thus to modulate membrane flux. Furthermore, the invention provides methods and compositions for expressing eukaryotic membrane proteins (e.g., proteins that mediate membrane flux) in particular unicellular organisms, and using these cells for screening assays to identify membrane protein-modulating agents.

One aspect of the invention provides nucleic acid constructs and nucleic acid compositions that encode one or more eukaryotic ion channel in prokaryotic cells. Examples of eukaryotic ion channels include calcium channels, potassium channels, sodium channels, chloride channels, and non-selective cation channels. Exemplary eukaryotic ion channels further include subunits, subtypes, isoforms, or homologs of any of the foregoing eukaryotic ion channels. A eukaryotic ion channel of the invention may also be a chimeric ion channel that is not a naturally occurring ion channel and comprises subunits or fragments from different naturally occurring ion channels (different subunits, subtypes, isoforms, or homologs of channels responsible for transport of the same ion, or channels responsible for transport of different ions).

In one embodiment, a nucleic acid construct of the present invention comprises a nucleic acid encoding a eukaryotic ion channel, a nucleic acid encoding a detectable marker, e.g., apo-aequorin, and at least one promoter. In one embodiment, the eukaryotic ion channel and the detectable marker are operably linked to a single promoter, and there may be intervening sequences between the nucleic acid encoding the eukaryotic ion channel and the nucleic acid encoding the detectable marker. In another embodiment, the eukaryotic ion channel is operably linked to a first promoter, and the detectable marker is operably linked to a second promoter. The first and second promoters may be the same, e.g., there are two separate promoters, and both are T7 promoters. Alternatively, the first and second promoters may be different, e.g., one is a T7 promoter and the other is a T3 promoter.

In one embodiment, a nucleic acid composition of the present invention comprises a nucleic acid construct encoding a eukaryotic ion channel suitable for expression in a prokaryotic cell. In one embodiment, the nucleic acid composition further comprises a second nucleic acid construct encoding a detectable marker, e.g., aequorin.

The invention contemplates the use of any promoter that operates to express an operably linked coding sequence in a prokaryotic cell. Certain embodiments may use well-known promoters for regulating expression in bacteria, such as a T3 or T7 promoter.

The invention also contemplates the use of any detectable marker that can indicate a change in ion channel activity. The detectable marker may be a polypeptide (e.g., aequorin), a luminescent marker, a colorometric marker (e.g., LacZ), or a small molecule (e.g., Fluo-4). The detectable marker can also be a biologically detectable event (e.g., the product of a biological event mediated by the ion channel of interest).

The invention further provides screening methods utilizing a prokaryotic cell of the invention. The screening methods are to identify, characterize, and/or optimize agents that modulate the activity of a membrane protein of interest expressed in the prokaryotic cells (e.g., that mediates ion flux). In one embodiment, the membrane protein is selected from an ion channel, a gap junction, a ionotropic receptor, and a transporter. The invention further provides screening methods utilizing a unicellular protist of the invention. The screening methods are to identify agents that modulate the activity of a membrane protein of interest expressed in the unicellular protist (e.g., that mediates ion flux). In one embodiment, the membrane protein is selected from an ion channel, a gap junction, a ionotropic receptor, and a transporter.

In one embodiment, the agent is an agonist of membrane protein of interest. That is, the agent potentiates biological activities of the membrane protein.

In another embodiment, the agent is an antagonist of the membrane protein of interest. That is, the agent inhibits biological activities of the membrane protein.

The invention further provides pharmaceutical compositions comprising an agent that is identified as a modulator of a membrane protein. Also provided are uses of the agent in manufacturing a medicament for modulating the activity of the membrane protein.

Further provided are methods of conducting a drug discovery business by carrying out the screening assays of the present invention, conducting therapeutic profiling of an identified membrane protein modulating agent for efficacy and toxicity, and formulating a pharmaceutical preparation with the modulating agent. The business method may further include establishing a distribution system and/or a marketing system for the pharmaceutical preparation.

### BRIEF DESCRIPTION OF THE DRAWINGS

FIG. 1 represents an example of a nucleic acid construct encoding a calcium ion channel and apo-aequorin. This vector allows for simultaneous expression in bacterial cells of both a channel protein, in this case the calcium channel human CatSper1, and a detectable marker, in this case apo-aequorin.

FIG. 2 is a graphic representation that demonstrates hCatSper-mediated calcium entry into its host bacteria cells as demonstrated by the aequorin-mediated optical response.

FIG. 3 shows dose response to calcium of hCatSper expressed in bacteria.

FIG. 4 shows control experiments using permeabilized bacteria cells. FIG. 4a shows that expression of a eukaryotic ion channel (hCatSper1) mediates calcium flux across a bacterial cell. FIG. 4b shows that this regulation of calcium flux is specific and is disrupted when the cells are permeabilized.

FIG. 5 shows CatSper1 and TRPV1 response to the calcium channel inhibitor, lanthanum. Calcium flux in bacterial cells mediated by expression of a eukaryotic calcium channel is inhibited in a dose dependent fashion following addition of lanthanum.

FIG. 6 shows a more detailed depiction of a vector for expressing both a channel protein and a detectable marker in bacterial cells. The dual-T7 promoters allow simultaneous expression of both the channel protein and the detectable marker.

FIG. 7 shows that eukaryotic channels expressed in bacterial cells specifically mediate ion flux (e.g., mediate flux of the same ions that the channel normally acts to modulate in eukaryotic cells). Expression of the eukaryotic calcium channel TrpV1 mediates calcium influx in bacterial cells. However, the Drosophila potassium channel Shaker (dShaker), which normally functions to modulate potassium flux, does not mediate calcium flux when expressed in bacterial cells.

FIG. 8 shows that rat TrpV1 mediates calcium flux when expressed in either CHO cells or bacterial cells. The Trp channel blocker ruthenium red inhibits TrpV1 mediated calcium flux in bacterial cells or CHO cells. Although this particular non-specific Trp inhibitor is somewhat less potent in bacterial cells, it is active. Additionally, although the IC50 differs, the calculated Hill slope values are not significantly different between the CHO cells and the bacterial cells.

FIG. 9 shows that two different eukaryotic calcium channels, hCatSper1 and rTrpV1, can be efficiently expressed in bacterial cells to produce functional calcium channels that mediate calcium flux across the bacterial cell.

Table 1 provides GenBank Accession Numbers for non-limiting examples of membrane protein nucleic acid and amino acid sequences.

### DETAILED DESCRIPTION OF THE INVENTION

The invention is based on the surprising finding that eukaryotic membrane proteins (e.g., proteins that mediate membrane potential), such as ion channels, can be expressed in prokaryotic cells. This finding provides novel methods and compositions for improving our understanding of membrane proteins, identifying and characterizing agents that can be used to modulate the activity of various membrane proteins, and identifying possible therapeutic agents for treating disorders and injuries related to the activity of membrane proteins in cells and tissues. Accordingly, the invention provides methods and compositions for expressing one or more eukaryotic membrane proteins in prokaryotic cells. The invention also provides prokaryotic cells that express one or more eukaryotic membrane proteins. The invention further provides methods and compositions for identifying an agent that can modulate the activity of one or more eukaryotic membrane proteins. The membrane protein-modulating agents, identified by methods of the invention, can be further used in making pharmaceutical compositions and manufacturing medicaments that can treat certain disorders or diseases by modulating a target's biological activity.

Additionally, the invention provides methods and compositions for expressing one or more eukaryotic membrane proteins in particular unicellular organisms. Exemplary unicellular organisms are unicellular protists, and exemplary unicellular protists include ciliated unicellular protists and unicellular protists containing one or more flagellum. One particular flagellated unicellular protist for use in the methods of the present invention is Chlamydomonas. These unicellular green algae contain a cell wall, a primitive “eye spot,” chloroplasts, and two flagella.

A. Nucleic Acid Constructs and Compositions

One aspect of the invention relates to nucleic acid constructs and compositions useful for expressing one or more eukaryotic membrane proteins in prokaryotic cells.

In one embodiment, a nucleic acid construct of the present invention comprises a nucleic acid sequence encoding a eukaryotic membrane protein. Exemplary eukaryotic membrane proteins include, but are not limited to, ion channels, gap junctions, ionotropic receptors, and transporter proteins. The invention contemplates that some functional membrane proteins are formed by expression of a single polypeptide chain, others are formed by homodimerization or homooligomerization (e.g., homotetramerization) of the same polypeptide chain or subunit, and still other functional membrane proteins are formed by heterodimerization or heterooligomerization (e.g., heterotetramerization) of the two or more different polypeptide chains or subunits. The invention contemplates the expression of one or more nucleic acids that constitute all or a portion of any of these functional membrane proteins.

In one embodiment, a nucleic acid construct of the present invention comprises a nucleic acid sequence encoding a eukaryotic ion channel and a nucleic acid sequence encoding a detectable marker, e.g., apo-aequorin.

In one embodiment, a eukaryotic ion channel and the detectable marker are operably linked to a single promoter. There may be intervening sequences between the nucleic acid encoding the eukaryotic ion channel and the nucleic acid encoding the detectable marker. The ion channel and the detectable marker may be expressed in tandem.

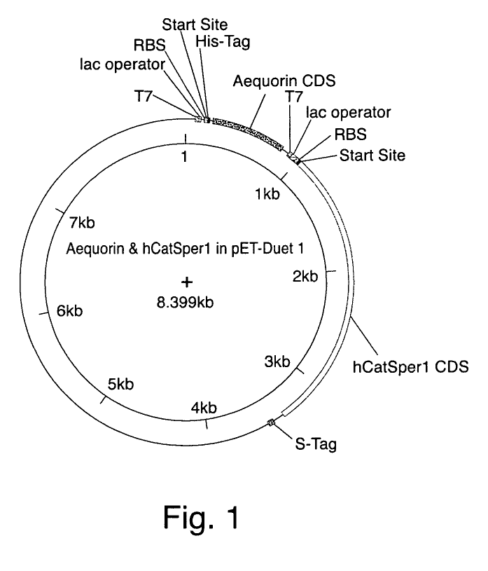

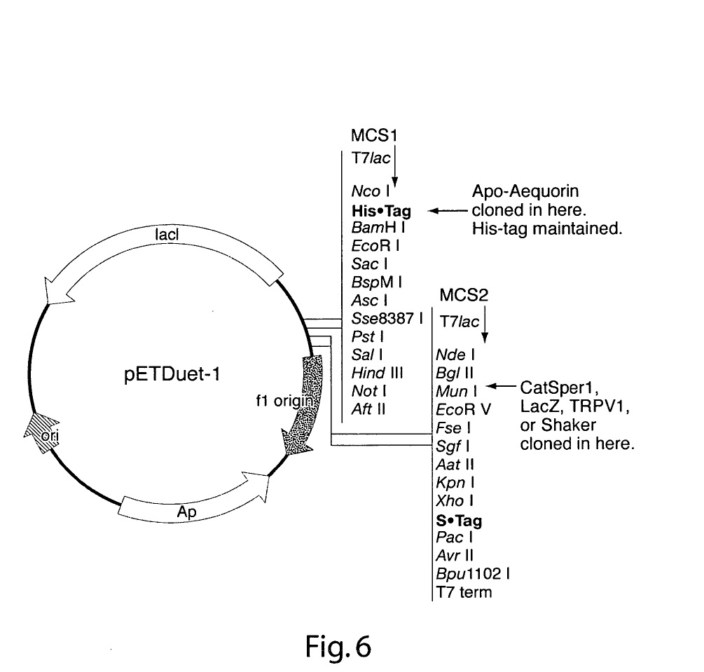

In another embodiment, the eukaryotic ion channel and the detectable marker are operably linked to a first promoter and a second promoter. The first and second promoters may be the same, e.g., both are T7 promoters. For example, a nucleic acid sequence encoding a eukaryotic ion channel and a nucleic acid sequence encoding apo-aequorin may each be cloned into one of the two multi-cloning sites (“MCS”) of the pETDuet™-1 vector (Novagen). Expression of each coding nucleic acid sequence will be driven from a T71ac promoter located before each MCS (see FIGS. 1 and 6). Alternatively, the first and second promoters may be different, e.g., one is a T7 promoter and the other is a T3 promoter.

In one embodiment, a nucleic acid composition of the invention comprises a nucleic acid construct encoding a eukaryotic ion channel suitable for expression in a prokaryotic cell. In one embodiment, the nucleic acid composition further comprises another nucleic acid construct encoding a detectable marker, e.g., apo-aequorin. The nucleic acid composition may comprise at least two separate vectors, each of which comprises a nucleic acid construct encoding the ion channel or the detectable marker. For example, a nucleic acid composition of the invention may comprise a first nucleic acid construct to express a eukaryotic ion channel in prokaryotic cells and a second nucleic acid construct to express a detectable marker in the same prokaryotic cells. Techniques are available in the art to introduce one or more nucleic acid constructs comprising one or more expression vectors into a host cell, e.g., a prokaryotic cell of the invention. The nucleic acid constructs can be introduced into the host cell simultaneously or sequentially.

Nucleic acid constructs of the present invention can encode any eukaryotic membrane protein to be expressed in a prokaryotic cell. For example, the nucleic acid construct can encode a eukaryotic ion channel, a gap junction, an ionotropic receptor, transporters, and the like. In one embodiment, the membrane protein is an ion channel and the ion channel may include, e.g., a calcium channel, a potassium channel, a sodium channel, a chloride channel, or a non-selective cation channel. Examples of ion channels are described in greater detail below.

In another aspect, the invention contemplates methods and compositions for expressing eukaryotic membrane proteins in unicellular organisms. In one embodiment, the unicellular organism is a protist. In another example, the unicellular organism is a ciliated protist. In another example, the unicellular organism is a flagellated protist. In still another example, the flagellated protist is Chlamydomonas.

The invention contemplates that all of the compositions and methods outlined in detail for the first aspect of the invention related to the expression of one or more eukaryotic membrane proteins in prokaryotic cells are similarly applicable to the expression of eukaryotic membrane proteins in unicellular organisms, such as unicellular protists. Furthermore the invention contemplates that all of the compositions and methods illustrated with reference to expression of ion channels (e.g., a particular class of membrane protein) are equally applicable to other classes of membrane proteins (e.g., gap junctions, transporters, ionotropic receptors, and the like). Furthermore, the invention contemplates that when more than one membrane protein is expressed in a cell (e.g., one or more, two or more, three or more, etc.) the one or more membrane protein may be of the same class (e.g., two or more ion channels) or of different classes (e.g., one or more ion channel and one or more ionotropic receptor).

In any of the foregoing, the invention contemplates the expression of one or more nucleic acid encoding all or a fragment or subunit of a functional membrane protein, as well as the expression of one or more nucleic acid encoding all or a fragment of two or more functional membrane proteins.

1. Membrane Proteins

The present invention provides methods and compositions for expressing eukaryotic membrane proteins in prokaryotic cells or in unicellular protists, and methods of using these compositions to identify agents that modulate the activity of one or more eukaryotic membrane protein. Exemplary classes of membrane proteins (e.g., proteins that mediate ion flux across the membrane) include ion channels, gap junctions, transporter proteins, and ionotropic receptors. By way of examples, particular members of each class of membrane proteins are described below. Such examples are provided merely to illustrate the characteristics of particular membrane proteins within the scope of the present invention.

a. Ion Channels

The invention contemplates methods and compositions for expressing, in prokaryotic cells, eukaryotic potassium channels, calcium channels, sodium channels, chloride channels, and non-selective cation channels which encompass, and are not limited to, G protein-gated potassium channels, inward rectifiers (usually potassium-selective), voltage-dependent calcium channels, voltage-independent calcium channels, calcium release-activated calcium channels, transient receptor potential (“TRP”) ion channels, sperm-specific calcium channels (e.g., CatSper isoforms 1-4), voltage-dependent sodium channels, voltage-independent sodium channels, Cystic Fibrosis transmembrane conductance regulator (“CFTR,” a chloride channel), CLC family chloride channels, extracellular ligand-gated ion channels, ATP-dependent channels, intracellular ligand-gated channels, cation non-selective channels, store-operated channels, etc.

By eukaryotic ion channels, the invention contemplates the use of all or a portion of an ion channel from any eukaryotic species. Exemplary eukaryotes include vertebrates and invertebrates. Exemplary eukaryotes include, but are not limited to, humans, mice, rats, cats, dogs, rabbits, sheep, cows, horses, goats, non-human primates, frogs, toads, fish, chicken, flies, worms, and yeast. In certain embodiments, the invention contemplates eukaryotic ion channels from vertebrates. In certain other embodiment, the invention contemplates eukaryotic ion channels from invertebrates. In certain other embodiment, the invention contemplates eukaryotic ion channels from mammals, reptiles, amphibians, or birds. In still other embodiment, the invention contemplates eukaryotic ion channels from plants. Eukaryotic ion channels also include chimeric ion channels comprising a portion of a eukaryotic ion channels and a portion of another eukaryotic ion channel (e.g., from the same eukaryotic species or from a different eukaryotic species), as well as a portion of a eukaryotic ion channel and a portion of a prokaryotic ion channel. Additionally, the invention contemplates the expression of one or more eukaryotic ion channel (e.g., one or more, two or more, three or more, etc.), as well as the expression of one or more eukaryotic ion channel and one or more membrane protein of another class (e.g., one or more gap junction, one or more ionotropic receptor, one or more transporter, etc.).

G protein-gated K⁺ channels (KGs) are found throughout the central nervous system and are activated by a large number of neurotransmitters including acetylcholine, adenosine, ATP, dopamine, GABA, opioids, cannabinoids, serotonin, norepinephrine, and somatostatin. KGs are heterotetrameric complexes formed by assembly of members of the GIRK (Kir 3.0) subfamily of inwardly-rectifying K⁺ channel subunits. Four mammalian GIRK (GIRK1-4) subunits have been identified, each containing a hydrophobic sequence common to all potassium-selective channels, two membrane-spanning domains, and cytoplasmic amino- and carboxyl-termini. The acetylcholine-gated atrial potassium channel IKACh is the prototypical member of the KG channel family. IKACh is formed by stoichiometric assembly of GIRK1 and GIRK4 subunits, and is a component of the parasympathetic signaling pathway that culminates in heart rate slowing.

Voltage-dependent Ca²⁺ channels enable excitable cells to dramatically increase cytosolic Ca²⁺ levels. Specialized Ca²⁺ trigger proteins, near the plasma membrane surface, initiate functions as diverse as exocytosis and contraction in neurons and muscle. In excitable cells depolarization from the resting membrane potential (˜−70 mV) initiates conformational changes in Ca²⁺-selective ion channels (Caᵥ) via special (S4) voltage-sensing regions of these molecules, catalyzing the flood of Ca²⁺ across the membrane. At ˜+150 mV the forces between chemical and electrical balance for Ca²⁺ are equal. Thus, at all physiological membrane potentials, Ca²⁺ flows into the cell. Voltage-dependent Ca²⁺ channel activity is self-limiting—the Ca²⁺ channel itself closes in a time-dependent fashion while further depolarization only decreases the electrochemical driving force for calcium entry.

In contrast to excitable cells, inexcitable cells enhance Ca²⁺ entry by hyperpolarization. These cells generally lack voltage-dependent Ca²⁺ channels. Open K⁺ channels force the membrane potential to more negative potentials, drawing Ca²⁺ more rapidly across the plasma membrane. Unlike excitable cells, Ca²⁺ enters through other specialized voltage-independent Ca²⁺-selective channels triggered by second messenger molecules. In essence, voltage-dependent Ca²⁺ channels drive rapid Ca²⁺ entry in excitable cells (e.g. neurons) while K⁺ channels drive Ca²⁺ entry in inexcitable cells. Ca²⁺ selectivity is insured by the structure of the channel pores which strain out all other ions. Ca²⁺ entering through voltage-dependent calcium channels may directly activate ryanodine receptors (RyR) to release Ca²⁺ from intracellular stores. Skeletal muscle is a specialized case of this theme in which dihydropyridine receptors on the surface of the plasma membrane and in T-tubules, directly abut the ER tetrameric RyR. Conformational changes induced by voltage in the dihydropyridine receptor result in Ca²⁺ influx and perhaps directly modulate the ryanodine receptor to release Ca²⁺ from intracellular stores.

One of the exciting areas in Ca²⁺ signal transduction in recent years has been the discovery of gating of Ca²⁺ entry by depletion of intracellular stores. Receptors such as muscarinic type 3 result in relatively rapid rises in intracellular [Ca²⁺] (Lechleiter et al., 1991a), effectively depleting endoplasmic reticular stores and somehow activating Ca²⁺ entry across the plasma membrane. Ca²⁺ entry through voltage-independent, La³⁺-sensitive pathways increases with hyperpolarization and dramatically alters the frequency of existing Ca²⁺ waves (Girard and Clapham, 1993).

Studies in the visual system have revealed the importance of cyclic nucleotide-gated ion channels (CNGs). These putative six-transmembrane channels pass calcium and sodium after activation by elevated levels of cyclic nucleotides including cAMP and cGMP. CNGs are divided into two classes: alpha and beta. Alpha subunits form functional conductances when expressed on their own. Beta subunits contribute to the channel pore and affect the affinity of the channel for cAMP and cGMP, but do not form functional conductances in the absence of alpha subunits. Mutations in CNGA3 or CNGB3 associate with achromotopsia. Retinitis pigmentosa, a common degenerative disease that causes blindness is caused my mutations in CNGA1, Outside the visual system, CNGA2 has been shown to be important in olfaction. Members of the CNG family include, but are not limited to CNGA1, CNGA2, CNGA3, CNGB1, CNGB2, and CNGB3. HCN channels are also modulated by cyclic nucleotides, but instead of sodium and calcium, they conduct potassium.

The Calcium-Release Activated Ca²⁺ channel, ICRAC, is a highly Ca²⁺-selective ion channel that is activated upon depletion of intracellular Ca²⁺ levels and depletion of intracellular Ca²⁺ stores. A 6 transmembrane domain-spanning protein (termed TRPV5; CaT1) exhibits some of the unique biophysical properties of ICRAC when expressed in mammalian cells. Like ICRAC, expressed TRPV5 is Ca²⁺-selective and inactivated by higher [Ca²⁺]ᵢ.

Transient receptor potential (TRP) ion channel subunit genes were first defined in the Drosophila visual system. In the trp mutation, the light response (receptor potential) decays during prolonged exposure to light. TRP-deficient flies are blinded by intense light because sustained Ca²⁺ entry via TRP ion channels and subsequent Ca²⁺-dependent adaptation is disrupted. Three genes (TRP, TRPL, TRPγ) in Drosophila encode TRP channels mediating fly vision and other unknown functions. Genetic approaches in flies have not resolved the mechanism of TRP activation, but confirm the importance of PLC-β and other components of the phosphatidylinositol pathway.

The mammalian TRP ion channels are encoded by more than 25 channel subunit genes. TRP channel primary structures predict six transmembrane (6TM) spanning domains with a pore domain between the fifth (S5) and sixth (S6) segments and both C and N termini located intracellularly. This architecture is a common theme for hundreds of ion channels present in life forms ranging from bacteria to mammals. The mammalian TRP channel family is united primarily by structural homology within the transmembrane spanning domains but overall sequence identities between members can be as low as 25%. Other features include a 25 amino acid motif (TRP domain) containing TRP box (EWKFAR) just C-terminal to the sixth transmembrane segment. TRP channels have been classified into at least six groups: TRPC (short), TRPV (vanilloid) and TRPM (long, melastatin), TRPP (polycystins), TRPML (mucolipins), and TRPA (ANKTM1). The invention contemplates the expression and use of TRPC, TRPV, TRPM, TRPA, TRPML and TRPP family members. The TRP domain and box are present in all TRPC channel genes, but not in all TRP channel genes. The N terminal cytoplasmic domain of TRPC, TRPV, and TRPA channels contain ankyrin repeats, while the TRPC and TRPM contain proline-rich regions in the region just C terminal to the predicted 6th TM segment.

The TRPC group can be divided into 4 subfamilies (TRPC1, TRPC4,5, TRPC3,6,7 and TRPC2) by sequence homology as well as functional similarities. Currently the TRPV family has 6 members grouped into 3 subfamilies. The TRPM family has 8 members divided into 4 groups. Constituents of the first subgroup include the founding member TRPM1 (Melastatin or LTRPC1) and TRPM3 (KIAA1616 or LTRPC3). TRPM7 (TRP-PLIK, ChaK(1), LTRPC7) and TRPM6 (ChaK2) belong to the second subgroup. TRPM2 (TRPC7 or LTRPC2) and TRPM8 (Trp-p8 or CMR1) form the third subgroup. TRPM5 (Mtr1 or LTRPC5) and TRPM4 (FLJ20041 or LTRPC4) constitute the fourth. The sole mammalian member of the TRPA family is ANKTM1. The TRPML family consists of the mucolipins, which include TRPML1 (mucolipins 1), TRPML2 (mucolipins 2), and TRPML3 (mucolipin3). The TRPP family consists of two groups of channels: those predicted to have six transmembrane domains and those that have 11. TRPP2 (PKD2), TRPP3 (PKD2 μl), TRPP5 (PKD2L2) are all predicted to have six transmembrane domains. TRPP1 (PKD1, PC1), PKD-REJ and PKD-1L1 are all thought to have 11 transmembrane domains. Though they have yet to be functionally expressed as channels, TRPP1 can form a novel conductance with TRPP2 in heterologous systems.

Mutations in both TRPP and TRPML family members are associated with diseases in humans. TRPP1 and TRPP2 were originally identified as the genes underlying two forms of polycystic kidney disease. Similarly, the TRPML1 was identified by positional cloning as the gene that underlies mucolipidosis type IV, a disease characterized by psychomotor and opthalmological abnormalities.

An unusual sperm Ca²⁺ channel has also been functionally characterized by the Clapham Laboratory at Children's Hospital, Harvard Medical School. The gene encoding this channel, termed CatSper, is unique in that it encodes a single 6 transmembrane-spanning repeat (like voltage-dependent Kᵥ channels) but its pore region and overall homology is closest to a single domain of the much larger four repeat Caᵥ. The gene product is exclusively expressed in the testis and not in other tissues such as the brain, heart, kidney or the immune system. At least four CatSper, 1-4, channels have been identified to date by the Clapham Laboratory and by the Garbers laboratory at University of Texas Southwestern. In sperm, the channel is localized primarily to the tail's principal piece, not the head or midpiece. Targeted disruption of the gene results in male sterility in otherwise normal mice. Sperm motility of the CatSper−/− (knockout) mice was dramatically decreased and they were unable to fertilize intact eggs. Cyclic AMP induced Ca²⁺ influx was abolished in the sperm of mutant mice. CatSper is thus crucial to cAMP mediated Ca²⁺ influx, sperm motility, and fertilization. CatSper represents an excellent target for non-hormonal contraceptives for both men and women. Recent findings also indicate that CatSper2 is also required for male fertility and sperm hyperactivation in mice.

Voltage-gated Na⁺ channels are crucial for the propagation of action potentials in excitable membranes. They cause the cell membrane to depolarize by allowing the influx of sodium ions into the cell. They have been known to cause this effect for almost half a century. Some 7000 sodium ions pass through each channel during the brief period (about 1 millisecond) that it remains open. However due to the size and hydrophobic nature of the channel protein, it has not been fully resolved by x-ray crystallography or NMR like the K⁺ channel.

Voltage-gated Na⁺ channels consist of an α-subunit responsible for selectivity and voltage gating. However some sodium channels also have one or two smaller subunits called α-1 and β-2. The voltage-gated sodium channel generally has 4 homologous domains containing multiple potential α-helical transmembrane segments. The segments are connected by non-conserved, hydrophilic intervening segments. The fourth transmembrane segment (S4) of each domain is highly positively charged, and thought to be a voltage sensor.

Chloride channels perform important roles in the regulation of cellular excitability, in transepithelial transport, cell volume regulation, and acidification of intracellular organelles. This variety of functions requires a large number of different Cl⁻ channels that are encoded by genes belonging to several unrelated gene families. The CLC family of chloride channels has nine known mammalian members that show a differential tissue distribution and function both in plasma membranes and in intracellular organelles. CLC proteins have about 10-12 transmembrane domains. They probably function as dimers and may have two pores. The functional expression of channels altered by site-directed mutagenesis has led to important insights into their structure-function relationship. Their physiological relevance is obvious from three human inherited diseases (myotonia congenita, Dent's disease and Bartter's syndrome) that result from mutations in some of their members and from a knock-out mouse model.

Chloride channels of the invention may also include CFTR, glutamate-gated Cl⁻ channel, glycine-inhibited Cl⁻ channel, glycine-gated Cl⁻ channel, histamine-gated Cl⁻ channel, intracellular calcium-activated Cl⁻ channel, voltage-gated Cl⁻ channel, etc.

The invention further contemplates the making and use of chimeric ion channels. A chimeric ion channel may comprise subunits or domains from different native channels. For example, subunits of different sodium channels may be swapped, or pore-forming domain and one or more transmembrane domains from two different sodium channels may be swapped. Another example is where subunits of a calcium channel and a sodium channel may be swapped, or a pore-forming domain of a calcium channel is swapped with a pore-forming domain of a sodium channel. The chimeric channels are particularly useful in studying different portions or domains of the channel proteins, for example, to elucidate the mechanisms underlying ion-selectivity or ligand-selectivity or gating mechanisms conferred by a portion or a domain of a channel protein of interest. The invention also contemplates interspecies chimeras. Interspecies chimeras include: chimeras wherein each subunit corresponds to a particular channel (e.g., the transmembrane domain of a particular human calcium channel+the pore forming domain of that same calcium channel derived from frog) or chimeras wherein each subunit corresponds to a different channel (e.g., the transmembrane domain of a particular human calcium channel+the pore forming domain of a particular frog sodium channel).

b. Gap Junctions

Cells are almost universally joined to each other by gap junctions comprised of low-resistance channels which allow intercellular diffusion of ions (electrical coupling) and small metabolites (metabolic coupling). Electrical coupling in electrically excitable tissues, such as myocardium, permits cell-to-cell passage of action potentials and the coordination of beating of the individual myocytes. The total range of functions of these low-resistance pathways between nonexcitable cells, such as between liver and lens cells, is not known, but the channels do permit cellular exchange of all small metabolites, second messengers, and ions.

Gap junction channels are composed of connexins, a family of integral membrane proteins which oligomerize into intercellular channels. A number of connexins have been implicated in human disease. Mutations in hCX26 are a major cause of non-autosomal deafness. In addition, mutations in connexin 32 correlates with X-linked Charcot-Marie-Tooth disease, while mutations in connexin 46 leads to some forms of cataracts. Mutations in a variety of connexins can associate with skin disorders. Proteins that form gap junctions include, but are not limited to Connexin 43, Connexin 46, Connexin 30, Connexin 26, Connexin 37, Connexin 40, Connexin 45, Connexin 50, Connexin 36, Connexin 59, Connexin 32, Connexin 30.3, Connexin 31, Connexin 31.3, Lens fiber MIP 17-20 and Lens fiber MIP 26.

The function of the connexins is being studied by targeted gene disruption in mice. In addition, the functions of intercellular communication in development are being studied by overexpression and dominant negative studies in Xenopus laevis. The present invention contemplates the expression of eukaryotic connexins to form gap junctions in prokaryotic cells or in unicellular protists. Cells expressing gap junctions can be used in methods to identify and characterize agents that modulate the activity of eukaryotic gap junctions. These gap junctional agonists and antagonists can be used and developed as pharmaceutical agents for modulating activity of gap junctions.

In contrast to tight and adherens junctions, gap junctions do not seal membranes together, nor do they restrict the passage of material between membranes. Rather, gap junctions are composed of arrays of small channels that permit small molecules to shuttle from one cell to another and thus directly link the interior of adjacent cells. Importantly, gap junctions allow electrical and metabolic coupling among cells because signals initiated in one cell can readily propagate to neighboring cells. In general, the upper limit for passage through gap junctions is roughly 1000 daltons (Da). Aside from ions, important examples of molecules that readily pass include cyclic AMP (329 Da), glucose-6-phosphate (259 Da) and nucleotides (250-300 Da).

Gap junctions are seen with an electron microscope as patches of varying size where the plasma membranes of neighboring cells are separated by a beautifully uniform gap, roughly 2-3 nm in width. The gap reflects areas of the two membranes that are connected by hexagonal tubes called connexins which form aqueous pores roughly 2 nm in diameter between the two cells. The major protein in purified preparations of gap junctions is connexin, which, when expressed in cells that normally do not have gap junctions, allows them to form. Different species of connexin are seen in different organisms and among different tissues within an organism. However, all connexins share a common structure of four membrane-spanning domains. A connexin is formed from six connexin molecules which extend a uniform distance outside the cells. Alignment of connexins from each cell across the gap results in the formation of the pores which functionally define the gap junction.

Gap junctions are dynamic structures because connexins are able to open and close. Elevated intracellular calcium and low intracellular pH are established stimuli for rapid closing of connexins. This may be of importance when one cell within a group becomes damaged—the idea is that closing the gap junctions in the damaged cell would effectively isolate that cell and prevent spreading of the injury.

Gap junctions are seen in virtually all cells that contact other cells in tissues. By way of example to illustrate the importance of gap junctions in normal and pathological physiology, gap junctions are abundant in cardiac and smooth muscle where they play an import role in electrical coupling. Depolarization of one group of muscle cells rapidly spreads to adjacent cells, leading to well-coordinated contractions of those muscles. In another example, gap junctions are important for the metabolic coupling of cells. Many hormones act by elevating intracellular concentrations of cAMP, which initiates a signalling pathway inside the cell. Cyclic AMP readily passes through gap junctions and thus, hormonal stimulation of one cell can lead to signal propagation to a cluster of cells.

c. Ionotropic Receptors

Ionotropic receptors are a large group of ligand gated ion channel. Unlike the voltage-gated channels which are classified by the type of ion they conduct, the ionotropic receptors are classified by ligand. These include nicotinic acetylcholine receptors, 5-HT3 receptors, glutamate receptors, glycine receptors, GABA receptors, and ATP-gated channels. Although ionotropic receptors contemplated by the present invention are not limited to those expressed in the central and peripheral nervous system, many well studied examples of ionotropic receptors endogenously function in these tissues. Accordingly, one preferred aspect of the present invention is the expression of such neuronal ionotropic receptors, and the use of the expressed receptors in screens to identify agents that can modulate the activity of one or more ionotropic receptor. Such agents may have particular use in methods of treating disorders and injuries of the central and peripheral nervous system.

Ionotropic receptors, also referred to as ligand-gated ion channels, act quickly to depolarize the neuron and pass on the action potential (or hyperpolarize the neuron and inhibit additional action potentials). These receptors are made up of five individual protein subunits embedded in the cell membrane, and arranged to form a single pore that spans this membrane. When a neurotransmitter associates with the extracellular recognition site, the membrane-spanning subunits of the receptor quickly open to form a pore through which the necessary ions can pass. Depolarization usually occurs a millisecond or two after the action potential has been received and lasts only up to ten milliseconds. For example, GABA is one example of a neurotransmitter recognized by an ionotropic receptor. GABA is an inhibitory neurotransmitter used at roughly one-third of the synapses in the brain. The binding of GABA at the GABA recognition site causes the membrane-spanning channel of the receptor protein to open and allow an influx of negatively charged chloride ions. This influx of negative ions serves to hyperpolarize the cell thus inhibiting the firing of an action potential. Though in the case of GABA, the ionotropic receptor is used to inhibit the firing of an action potential, there are other ionotropic receptors which recognize excitatory neurotransmitters and thus stimulate the firing of action potentials in post-synaptic cells.

By way of further nonlimiting example, other ionotropic receptor families include the nicotinic receptor family, glutamate receptor family, and the ATP receptor family. The nicotinic receptor family includes nACh receptors (Na⁺/K⁺ channel), GABA_{A} and GABA_{C} receptors (Cl⁻ channels), Glycine receptors (Cl⁻ channel), and 5-HT₃ receptor (Na⁺/K⁺ channel). Representative examples of these nicotinic receptor family members include GABA receptor-alpha 1, GABA receptor-alpha 2, GABA receptor-alpha 3, GABA receptor-beta 1, GABA receptor-beta 2, GABA receptor-beta 3, GABA receptor-delta, GABA receptor-gamma 2, GABA receptor-rho 1, GABA receptor-rho 2, GABA receptor-rho 3,5-hydroxytryptamine (serotonin) receptor 3A, and the solute carrier family 6 (glycine) neurotransmitter. ATP-gated receptors include P2X₁, P2X₂, P2X₃, P2X₄, P2X₅, P2X₆ P2X₇ among others.

To further illustrate the potential use of ionotropic receptors to identify agents that can modulate their activity and then to use such agents to design therapeutics, we note that agents that modulate the activity of GABA have been identified. In 1957, quite by accident, scientists discovered that an unknown compound reduced fear in animals. When tested on humans with anxiety disorders, this drug, called Librium, was found to be a powerful tranquilizer. Since then, many other drug companies have made minor chemical changes in the structure of Librium, producing a large number of similarly-shaped drugs called benzodiazepines. Two well known benzodiazepines are valium and xanax, tranquilizers which work to reduce fear and anxiety. Although these drugs do not bind to the GABA recognition site, they bind to a second site on the GABA receptor.

Because they do not bind to the GABA recognition site, the benzodiazepines do not work as directly as GABA in allowing the free passage of chloride ions through the pore of the receptor. Instead, when a drug like valium or xanax binds to its specific recognition site, the 3-dimensional shape of the GABA receptor changes in such a way as to allow GABA to bind more easily to its own binding site. Increased GABA-binding leads to an increased influx of chloride ions at the post-synaptic cell. Whether this increase in chloride influx is due to an increase in the amount of time that the channel stays open, or to a greater affinity for the GABA molecule at its binding site is not currently known. It is, however, clear that increased chloride influx causes an increased number of inhibitory inputs at the post-synaptic neuron. This increase in inhibitory inputs makes it difficult for any excitatory inputs to cause an action potential in the post-synaptic cell. When the excitatory signals caused by fear or stress are inhibited in this way, the result is a marked decrease in anxiety and a greater feeling of calm.

The finding that benzodiazepines bind to the GABA receptor raised the question of whether there were endogenous neurochemical(s) shaped like the benzodiazepines, which could bind to the same spot on the GABA receptor. Such a compound was subsequently identified in the brains of rats and in the cerebral spinal fluid of humans. This chemical, a peptide named diazepam binding inhibitor (DBI), was indeed, found to bind to the same site as the benzodiazepines, yet caused an effect that was opposite to these tranquilizers. Upon binding, DBI changes the conformation of the GABA receptor such that the ability of GABA to bind to its receptor site is decreased. This leads to a decrease in the influx of chloride ions into the postsynaptic cell and, as a result, fewer inhibitory post-synaptic potentials. DBI is thus a negative modulator of the GABA receptor.

In order to determine the behavioral effect of this endogenous peptide, neuroscientists worked on developing synthetic compounds which would bind to the same recognition site as DBI and which, like DBI, would act as negative modulators of the GABa receptor. The production of compounds which came to be known as “inverse agonists” have helped confirm the role of DBI as an endogenous anxiogenic. When injected into human volunteers, one of these synthetic inverse agonists, FG7142, caused sweating, nausea, palpitations, tightness in the chest, restlessness, tremors, and feelings of worry and impending doom—all markers of increased anxiety. Some people are naturally more anxious than others. Such behavioral variation most likely results from individual differences in the steady state production of DBI, and thus the amount of this peptide normally found in the brain. The steady state production of DBI is thought to be a result of genetic background, as well as early conditioning experiences.

It has recently been discovered that certain steroids also have a binding site on the GABA receptor. This binding site is again distinct—it is not the place where GABA binds, nor is it the place where DBI and the benzodiazepines bind. The steroids that bind at these sites are unique in that they are synthesized from circulating cholesterol locally in the glial cells of the brain. In times of stress, cholesterol is converted to progesterone, which is then converted to allopregnanolone. Because of its direct effect on neurotransmission, allopregnanolone is called a neuroactive steroid. Like the benzodiazepines, this hormone acts as a positive modulator of the GABA receptor, increasing the ability of GABA to bind to the receptor, and thus increasing the influx of chloride ions at the post-synaptic membrane. A second neuroactive steroid is a progesterone precursor known as pregnenolone. This neuroactive steroid differs from allopregnanolone in that it is a negative modulator of the GABA receptor, thus producing the same effects as DBI.

A second class of ionotropic receptors are glutamate receptors. Glutamate is the major excitatory neurotransmitter in the central nervous system. Excessive glutamate neurotransmission underlies ischaemia, excitotoxicity, and neurodegeneration. Biophysical analysis of membrane patches taken from neuronal cell bodies indicate that different glutamate-sensitive channel types co-exist in many cells.

Three glutamate receptor types have been identified: AMPA receptors (GluR1-4), Kainate receptors (GluR5-7, KA1, KA2), and NMDA receptors (NR1, NR2A-D). AMPA α-amino-3-hydroxy-5-methyl-4-isoxazolepropionate) sensitive receptors, kainate (neurotoxin) sensitive receptors, and NMDA (N-methyl-D-aspartate) sensitive receptors differ in structure from nicotinic family receptors. The glutamate receptor polypeptide has 4 hydrophobic regions, 3 transmembrane spanning domains, and 1 loop segment (region 2) which inserts into the membrane. Mutagenesis studies have indicated that a specific amino acid residue in region 2 can determine receptor ion permeability. Amongst AMPA/kainate receptors, this residue is the glutamine/arginine (Q/R) site. Amongst NMDA receptors, this residue is an asparagine (N).

d. Ion Transporters

Ion transporters differ from ion channels in that they require energy. This usage of ATP enables transporters to pump ions against an electrochemical gradient. In addition to regulating the intracellular concentrations of rare ions such as iron, ion transporters also maintain the electrochemical gradient across the plasma membrane and prevent “run down”. Families of ion transporters include the ATP-Binding Cassette family and the solute carrier family. These families contain hundreds of different proteins that transport or exchange various substances across the cell membrane. Transporters that cause ion flux include, but are not limited to, sodium-potassium ATPases, sodium-hydrogen exchangers, sodium-calcium exchangers, sodium-iodide symporters, manganese transporters, zinc transporters, iron transporters, magnesium transporters, potassium-chloride transporters, sulfonyl urea receptor and anion transporters.

Mutations in ion transporters can cause a variety of diseases. DMT1 mutations can cause severe anemia. Ion transporters are also verified pharmaceutical targets. The commonly used drug digitalis which increases the strength of heart contractions works through the sodium-potassium ATPase.

2. Nucleic Acid Constructs and Compositions

The present invention also relates to nucleic acid constructs and compositions useful to express eukaryotic membrane proteins in prokaryotic cells (e.g., to express one or more, two or more, three or more, etc. membrane protein). The invention further relates to nucleic acid constructs and compositions useful to express eukaryotic membrane proteins in unicellular organisms, such as unicellular protists (e.g., to express one or more, two or more, three or more, etc. membrane protein).

The term “membrane protein” refers to a transmembrane protein that mediates ion flux across a cellular membrane. The terms “membrane protein” and “protein that mediates ion flux” and “protein that mediates membrane flux” are used interchangeably throughout the application. Exemplary classes of membrane proteins are ion channels, gap junctions, transporter proteins, and ionotropic receptors, and many specific membrane proteins exist within each of these classes. The invention contemplates methods and compositions for expressing any of these eukaryotic membrane proteins (e.g, a membrane protein from any of these classes of membrane proteins) in a prokaryotic cell or in a unicellular protist.

By eukaryotic membrane protein, the invention contemplates the use of all or a portion of a membrane protein from any eukaryotic species. By way of example, in certain aspects, the invention contemplates methods and compositions related to eukaryotic ion channels. Exemplary eukaryotes include vertebrates and invertebrates. Exemplary eukaryotes include, but are not limited to, humans, mice, rats, cats, dogs, rabbits, sheep, cows, horses, goats, non-human primates, frogs, toads, fish, chicken, flies, worms, and yeast. In certain embodiments, the invention contemplates eukaryotic ion channels from vertebrates. In certain other embodiments, the invention contemplates eukaryotic ion channels from invertebrates. In still other embodiments, the invention contemplates eukaryotic ion channels from mammals, reptiles, amphibians, fish, or birds. In still another embodiment, the invention contemplates eukaryotic membrane proteins from plants.

Additionally, we note that by “eukaryotic ion channel” or “eukaryotic membrane protein” is meant that the expressed protein itself contains all or a portion of a eukaryotic ion channel or a eukaryotic membrane protein. Accordingly, the invention contemplates that the term “eukaryotic membrane protein” includes a eukaryotic membrane protein expressed using either prokaryotic or eukaryotic promoters and/or expression elements. Furthermore, the term “eukaryotic membrane protein” encompasses chimeric proteins containing a portion of a eukaryotic membrane protein and a portion of a different eukaryotic membrane protein, as well as chimeric proteins containing a portion of a eukaryotic membrane protein and a portion of a prokaryotic membrane protein. As long as a portion of the protein is eukaryotic, the chimeric protein is classified as a eukaryotic membrane protein within the scope of the present invention.

A nucleic acid construct or composition of the present invention generally comprises an expression vector which comprises a coding sequence for a eukaryotic membrane protein (e.g., including, but not limited to, a coding sequence for a portion or a subunit of a functional membrane protein that homo or heterodimerizes to produce the functional membrane protein). The invention contemplates the use of nucleic acid sequences corresponding to the nucleic acid sequences that encode a eukaryotic membrane protein derived from any eukaryotic species. Furthermore, the invention contemplates the use of nucleic acid sequences that differ due to the degeneracy of the genetic code but that encode a eukaryotic membrane protein. Still furthermore, the invention contemplates the use of nucleic acid sequences that differ due to allelic differences, silent mutations, cloning artifacts and the like, to the nucleic acid sequences that encode eukaryotic membrane proteins. Exemplary sequences for use in the methods of the present invention include nucleic acid sequences that hybridize under stringent conditions, including a wash step of 0.2×SSC at 65° C., to a nucleic acid sequence that encode all or a subunit of a functional eukaryotic membrane protein.

Optionally, a nucleic acid construct or composition of the present invention may further comprise an expression vector which comprises a coding sequence for a detectable marker. A “vector” is a replicon, such as plasmid, phage or cosmid, to which another DNA segment may be attached. The term “vector” refers to a nucleic acid molecule capable of transporting another nucleic acid to which it has been linked. One type of vector is an episome which is a nucleic acid capable of extra-chromosomal replication. Vectors capable of autonomous replication and/or expression of nucleic acids to which they are linked may also be used. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. However, the invention is intended to include such other forms of expression vectors which serve equivalent functions and which become known in the art subsequently hereto. Examples of vectors that can be used to practice the invention include, but are not limited to, pET vector (e,g., for expression in bacteria, see FIG. 1 for a modified pET vector), pCrGRP (e.g., for expression in GC-rich organisms, e.g., Chlamydomonas), pSSCR7 (a 4.3 kbp vector for nuclear expression in Chlamydomonas constructed by the insertion of beta-2 tubulin promoter, Chlamydomonas cell membrane protein terminator, and multicloning sites into pUC18), etc.

A DNA or nucleic acid “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence of the present invention can include, but is not limited to, cDNA from eukaryotic mRNA, genomic DNA sequences from eukaryotic (e.g., mammalian) DNA, and synthetic DNA sequences. A polyadenylation signal and transcription termination sequence may be located 3′ of the coding sequence.

Nucleic acid or DNA regulatory sequences or regulatory elements are transcriptional and translational control sequences, such as promoters, enhancers, polyadenylation signals, and terminators, that provide for and/or regulate expression of a coding sequence in a host cell. Regulatory sequences for directing expression of eukaryotic ion channels and detectable markers of certain embodiments are art-recognized and may be selected by a number of well understood criteria. Examples of regulatory sequences are described in Goeddel, Gene Expression Technology: Methods in Enzymology (Academic Press, San Diego, Calif. (1990)). For instance, any of a wide variety of expression control sequences that control the expression of a DNA sequence when operatively linked to it may be used in these vectors to express DNA sequences encoding the ion channels and detectable markers. Such useful expression control sequences, include, for example, the early and late promoters of SV40, beta2 tubulin, adenovirus or cytomegalovirus immediate early promoter, the lac system, the trp system, the TAC or TRC system, T7 promoter whose expression is directed by T7 RNA polymerase, the promoter for 3-phosphoglycerate kinase or other glycolytic enzymes, the promoters of acid phosphatase, e.g., Pho5, and the promoters of the yeast α-mating factors and other sequences known to control the expression of genes of prokaryotic or eukaryotic cells or their viruses, and various combinations thereof. It should be understood that the design of the expression vector may depend on such factors as the choice of the host cell to be transformed. Moreover, the vector's copy number, the ability to control that copy number and the expression of any other protein encoded by the vector, such as antibiotic markers, should also be considered.

The invention contemplates the use of any promoter that can drive the expression of a eukaryotic membrane protein in prokaryotic cells or unicellular protists. As used herein, the term “promoter” means a DNA sequence that regulates expression of a selected DNA sequence operably linked to the promoter, and which effects expression of the selected DNA sequence in cells. A “promoter” generally is a DNA regulatory element capable of binding RNA polymerase in a cell and initiating transcription of a coding sequence. For example, the promoter sequence may be bounded at its 3′ terminus by the transcription initiation site and extend upstream (5′ direction) to include the minimum number of bases or elements necessary to initiate transcription at levels detectable above background. Within the promoter sequence may be found a transcription initiation site, as well as protein binding domains responsible for the binding of RNA polymerase. Eukaryotic promoters will often, but not always, contain “TATA” boxes and “CAAT” boxes. Various promoters, including inducible promoters, may be used to drive the various vectors of the present invention.

The term “promoter” also encompasses prokaryotic and/or eukaryotic promoters and promoter elements. Given that certain embodiments of the present invention are performed in prokaryotic cells, the invention contemplates that the use of prokaryotic and/or eukaryotic promoters, enhancers, or other elements may be useful. One of skill in the art can select from amongst eukaryotic and prokaryotic promoters, promoter elements, and enhancers to select the combinations of elements appropriate to express a given membrane protein in a given cell. The invention contemplates the use of (i) prokaryotic promoters and elements in prokaryotic cells, (ii) prokaryotic promoters and elements in unicellular protists, (iii) eukaryotic promoters and elements in prokaryotic cells, (iv) eukaryotic promoters and elements in unicellular protists, (v) a combination of prokaryotic and eukaryotic promoters and elements in prokaryotic cells, and (vi) a combination of prokaryotic and eukaryotic promoters and elements in unicellular protists.

The term “promoter” as used herein encompasses “cell specific” promoters, i.e. promoters, which effect expression of the selected DNA sequence only in specific cells (e.g., cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e., expression levels can be controlled).

3. Detectable Markers

Certain embodiments of the invention comprise a detectable marker that can indicate the activity of a protein that mediates ion flux. In one embodiment, the protein that mediates ion flux across the membrane (e.g., membrane protein) is an ion channel. In another embodiment, the protein that mediates ion flux across the membrane is a gap junction. In another embodiment, the protein that mediates ion flux across the membrane is a transporter. In still another embodiment, the protein that mediates ion flux is an ionotropic receptor.

The detectable marker can be an expressable polypeptide, however, the invention similarly contemplates the use of any detectable marker (e.g., a chemical reagent, radioactive reagent, luminescent reagent, fluorescent reagent, etc.). Provided are nucleic acid constructs that can express such polypeptides, for example, aequorin (or apoaequorin). Aequorin is the bioluminescent protein complex isolated from the jellyfish Aequorea victoria. The protein is abundant in the jellyfish and the purified and crystallized forms of the protein have been extensively studied for many years. The bioluminescent complex is composed of a protein, termed apoaequorin, and a chromophore cofactor, coelenterazine. Several isoforms of apoaequorin and many analogues of coelenterazine are available. Combinations of these components form complexes with a wide range of [Ca²⁺] sensitivities. The mechanism of photon emission in the presence of elevated [Ca²⁺] is well understood from the crystal structure and from other studies. Binding of aequorin with trace of amounts of free Ca²⁺ results in oxidation of coelenterazine, and yields apoaequorin, coelenteramide, CO₂, and light. Once the photon has been emitted, the complex must dissociate and the apoaequorin must combine with an activatable cofactor (e.g., coelenterazine) before the complex can emit another photon. The following references describe apoaequorin in greater details: O. Shimomura, F. H. Johnson, Y. Saiga, J. Cell. Comp. Physiol. 59, 223 (1962); O. Shimomura and F. H. Johnson, Biochemistry 8, 10, 3991 (1969); O. Shimomura and F. H. Johnson, Nature 256, 236 (1975); O. Shimomura and F. H. Johnson, Proc. Nat. Acad. Sci. U.S.A. 72, 4, 1546 (1975); O. Shimomura and F. H. Johnson, Proc. Nat. Acad. Sci. U.S.A. 75, 6, 2611 (1978); O. Shimomura and A. Shimomura, Biochem. J. 199, 825 (1981); O. Shimomura, Society for Experimental Biology 351 (1985).

Cloned DNAs encoding aequorin have been expressed in bacteria and produced a functional bioluminescent complex when bacterial extracts were combined with the coelenterazine cofactor (M. R. Knight, A. K. Campbell, S. M. Smith, A. J. Trewavas, FEBS 282, 2, 405 (1991); S. Inouye, Y. Sakaki, T. Goto, F. I. Tsuji, Biochemistry 25, 8425 (1986). Subsequently other studies have shown that the cDNA can direct the synthesis of the functional apoprotein in plants and yeast as well (M. R. Knight, A. K. Campbell, S. M. Smith, A. J. Trewavas, Nature 352, 524 (1991); J. Nakajima-Shimada, H. Lida, F. I. Tsuji, Y. Anraku, Proc. Natl. Acad. Sci. U.S.A. 88, 6878 (1991)). EPO Application 0 341 477 also taught the expression of aequorin in a mammalian cell system.

Detectable markers other than expressable polypeptides can also be used, which include small molecule ion indicators. The invention contemplates a wide range of indicators including, but not limited to, chemical, luminescent, fluorescent, radioactive, and the like. Examples of ion indicators include, but are not limited to, the following indicators:

1) Ca²⁺ indicators: Fura-2 (e.g, 1-[6-Amino-2-(5-carboxy-2-oxazolyl)-5-benzofuranyloxy]-2-(2-amino-5-methylphenoxy) ethane-N,N,N′,N′-tetraacetic acid, pentapotassium salt), Indo-1 (e.g, 1-[2-Amino-5-(6-carboxy-2-indolyl)phenoxy]-2-(2-amino-5-methylphenoxy)ethane-N,N,N′,N′-tetraacetie acid, pentapotassium salt), Fluo-3 (e.g, 1-[2-Amino-5-(2,7-dichloro-6-hydroxy-3-oxy-9-xanthenyl)phenoxy]-2-(2′-amino-5′-methylphenoxy)ethane-N,N,N′,N′-tetraacetic acid), Fluo-4, Rhod-2 (e.g., 1-[2-Amino-5-(3-dimethylamino-6-dimethylammonio-9-xanthenyl)phenoxy]-2-(2-amino-5-methylphenoxy)ethane-N,N,N′,N′-tetraacetic acid, chloride), BAPTA (e.g, 1,2-bis(o-aminophenoxy)ethane-N,N,N′,N′-tetraacetic acid), Quin-2 (e.g, 8-Amino-2-[(2-amino-5-methylphenoxy)methyl]-6-methoxyquinoline-N,N,N′,N′-tetraacetic acid, tetraacetoxymethyl ester), calcium orange, calcium crimson, calcium green.

2) pH indicators: BCECF (e.g, bis-(carboxyethyl)-5-(and-6)-carboxyfluorescein), Caboxylfluorescein.

3) Mg²⁺ indicators: Furaptra (also known as Mag-Fura-2), Indo-magnesium.

4) Na⁺/K⁺ indicators: SBFI, PBFI.

5) Cl⁻ indicators: SPQ (6-methoxy-N-(3-sulfopropyl)quinolinium), MQAE (e.g, N-(Ethoxycarbonylmethyl)-6-methoxyquinolinium bromide), MEQ (e.g., 6-Methoxy-N-ethylquinolinium iodide.

6) Zn²⁺ indicators: TSQ (e.g., N-(6-methoxy-8-quinolyl)-p-toluenesulfonamide).

7) Fluorophores: fluorescein, carboxylfluorescein, dichlorocarboxylfluorescein, carboxy rhodamine and their succinimydyl esters.

Also provided are leakage resistant indicators: Fura PE-3, Indo PE-3, and Fluo-LR. Also provided are high Ca²⁺ indicators: Fura-2FF, Indo-1FF, and Fluo-3FF. Also provided are near membrane calcium indicators: FFP18, FIP18, and Fluo-FP18. Also provided are long-wavelength calcium indicators: KJM-1. KJM-1FF, Fluo533, Fluo535-FF. Also provided are other special indicators: pKc, Zinc and sodium ionophores.

Ion indicators of the invention may also be a fluorescent dye, and suitable dyes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue.TM., Texas Red, and others described in the 6th Edition of Molecular Probes Handbook by Richard P. Haugland.

Indicators of ion flux may also include FRET based dyes that track membrane potential. Suitable dyes include, but are not limited to, coumarin-labeled phospholipids (CC2-DMPE), bis-(1,3-dialkylthiobarbituric acid) trimethine oxonol, DisBac (see, Gonzalez and Maher, Receptors and Channels 8).

The invention is intended to encompass all custom-synthesized ion indicators, as well as indicators to be discovered. Further provided are free acid form or different salt forms of the ion indicators, e.g., as listed above. For example, the salt forms can be AM ester, pentaammonium, pentapotassium, pentasodium, tetraammonium tetrapotassium, tetrasodium, triammonium, tripotassium, trisodium, etc.

The invention is also designed to encompass ion flux measured by use of radioactive ions. For example, the activity of a membrane protein that allows calcium to permeate the cell could be accomplished by measuring uptake of Ca⁴⁵. By way of further example, one of skill in the art can use rubidium flux to assay activity of potassium channels.

The invention further contemplates using a biologically detectable event as a detectable marker. A biologically detectable event means an event that changes a measurable property of a biological system, for example, without limitation, light absorbance at a certain wavelength, light emission after stimulation, presence/absence of a certain molecular moiety in the system, electrical resistance/capacitance etc., which event is conditional on another, possibly non-measurable or less easily measurable property of interest of the biological system, for example, without limitation, the presence or absence of an interaction between two proteins. Preferably, the change in the measurable property brought about by the biologically detectable event is large compared to natural variations in the measurable property of the system. Examples include the yellow color resultant from the action of β-galactosidase on o-nitrophenyl-b-D-galactopyranoside (ONPG) (J. H. Miller, Experiments in Molecular Genetics, 1972) triggered by transcriptional activation of the E. coli lacZ gene encoding β-galactosidase by signaling mediated by the eukaryotic membrane protein. Another example of biologically detectable event of this invention involves Ca²⁺-dependent protein phosphorylation, a phenomenon well-studied in muscle cells and recently known to be present in prokaryotes. For example, Elizarov and Danielenko reported Ca²⁺-dependent protein phosphorylation in Streptomyces fradiae as well as Ca²⁺-induced alterations in intensity and specificity of the secondary metabolism and stimulation of aerial mycelium formation and sporulation (FEMS Microbiol. Lett. 202(1): 135-8, 2001). Polypeptide indicators of ion concentration and membrane potential also detect biological events. Examples of such indicators include calcium sensing chameleons (Miyawaki et al., 1997, Nature 388). Other examples of biologically detectable events are readily apparent to the person skilled in the art. Alternatively, other biological functions may be induced and detected following ion entry into or exit from a cell. For example, transcriptional regulation, secondary modification, cell localization, exocytosis, cell signaling, protein degradation or inactivation, cell viability, regulated apoptosis, growth rate, cell size. Such biological events may also be controlled by a variety of direct and indirect means including particular activities associated with individual proteins such as protein kinase or phosphatase activity, reductase activity, cyclooxygenase activity, protease activity or any other enzymatic reaction dependent on subunit association. Also, one may provide for association of G proteins with a receptor protein associated with the cell cycle, e.g. cyclins and cdc kinases, or multiunit detoxifying enzymes.

B. Prokaryotic Cells and Unicellular Protists of the Invention

The invention further provides prokaryotic cells and unicellular protists that can express or express one or more eukaryotic membrane protein. For example, the invention provides prokaryotic cells or unicellular protists that can express any of a number of eukaryotic membrane proteins that mediate ion flux including ion channels, gap junctions, ionotropic receptors, transporters, and the like.

Prokaryotic cell is defined as a cell that lacks a membrane-bonded nucleus, does not undergo meiosis, and lacks the structurally complex chromosomes found in eukaryotes. The invention is intended to encompass any type or form of prokaryotic cell that can be used to as a host cell to express a eukaryotic ion channel.

Certain embodiments comprise a bacterium cell that expresses a eukaryotic ion channel. More specifically, the invention encompasses an E. coli cell expressing a eukaryotic ion channel. The E. coli cell may further comprise a nucleic acid sequence encoding a detectable marker that can indicate activity of the eukaryotic ion channel. The invention further contemplates utilizing modified bacteria strains. For example, an E. coli strain named “Rosetta-Gami B (DE3) pLysS” from Novagen may be used as a host cell to express a eukaryotic ion channel. The invention contemplates, however, that any prokaryotic cell can be adapted to the methods and compositions of the present invention.

The prokaryotic cells of the invention are useful in various ways. For example, these cells may be used as expression systems to make recombinant eukaryotic ion channels. Moreover, these cells may provide an important component in establishing screening assays for identifying an agent that modulates activities of the eukaryotic ion channels expressed or to be expressed in these cells. These cells can also serve as study models to examine the function of channel subunits or domain structure, e.g., by making chimeric ion channels of the invention.

In addition to prokaryotic cells, the invention contemplates that certain unicellular organisms may be suitable for the methods and compositions of the present invention. In one embodiment, the invention contemplates the use of unicellular protists.

Certain unicellular protists possess additional characteristics that may make them especially suitable for the methods and compositions of the invention. See, e.g., Harris, The Chlamydomonas Sourcebook: A Comprehensive Guide to Biology and Laboratory Use (Academic Press, 1989); The Molecular Biology of Chloroplasts and Mitochondria in Chlamydomonas, (Rochaix et al. eds., Kluwer Academic Publishers, Dordrecht, 1998). Specifically, ciliated protists and protists possessing one or more flagella may be useful for the methods and compositions of the present invention. In one embodiment, the invention contemplates methods and compositions in which one or more proteins that mediates ion flux (e.g., a membrane protein) are expressed in a ciliated protist. In another embodiment, the invention contemplates methods and compositions in which one or more proteins that mediates ion flux (e.g., a membrane protein) are expressed in a flagellated protist. In still another embodiment, the flagellated protist is Chlamydomonas.

Similarly, the unicellular protists of the invention are useful in various ways. For example, these cells may be used as expression systems to make recombinant eukaryotic ion channels. Moreover, these cells may provide an important component in establishing screening assays for identifying an agent that modulates activities of the eukaryotic ion channels expressed or to be expressed in these cells. These cells can also serve as study models to examine the function of channel subunits or domain structure, e.g., by making chimeric ion channels of the invention.

C. Screening Methods

Thus provided are methods of screening for and identifying, characterizing, and or optimizing agents that can modulate the activity of a membrane protein. Exemplary membrane proteins include ion channels, gap junctions, ionotropic receptors, transporters, and the like. The term “modulate” as used herein refers to both “upregulate” (i.e., activate or stimulate (e.g., by agonizing or potentiating)) and “downregulate” (i.e. inhibit, block or suppress (e.g., by antagonizing, decreasing, or inhibiting)). Ion flux can be a biological function directly mediated by the membrane protein. In one example, the membrane protein is an ion channel and the biological activity mediated by the ion channel includes, e.g., the ability of the channel to conduct ion flux into or out of a cell. Alternatively, activity can be a measure of quantity of the protein present in a cell, which may depend on the expression of the protein in the cell and/or the amount of expressed protein properly assembled in the cell membrane.

The prokaryotic cells of the invention can express one or more eukaryotic membrane protein. The expressed eukaryotic protein(s) are screened for activation or blockage by test compounds. Thus, a method of identifying, characterizing, and/or optimizing an agent that modulates ion flux involves expressing in a prokaryotic host cell a eukaryotic membrane protein, or a homolog, variant, or fragment thereof capable of forming a pore or conferring ion selectivity or another functional aspect of the membrane protein; contacting the host cell with an agent; and measuring the effect of the agent on the activity of the protein.

The activity of a protein that mediates ion flux can be measured using any of a number of methods. For example, ion channel activity can be measured by various assays including high throughput screening assays, radioactive ion flux assays (Ca²⁺ uptake, rubidium flux), electrophysiological assays (e.g., patch clamp and patch clamp arrays), fluorescence changes (e.g., voltage-sensitive dyes and Ca²⁺-sensitive dyes), FRET assays (including those using fluorescent dyes and polypeptide indicators), contractile behavior, secretion, enzyme (e.g., kinase or phosphatase) activity, and various binding assays (e.g., ion flux assay, a membrane potential assay, a yeast growth assay, a cAMP assay, an inositol triphosphate assay, a diacylglycerol assay, an Aequorin assay, a Luciferase assay, a FLIPR assay for [Ca²⁺]ᵢ, a mitogenesis assay, a MAP Kinase activity assay, an arachidonic acid release assay, and an assay for extracellular acidification rates, etc.). The invention further contemplates an agent discovered by screening agents using the above method.

The invention also contemplates high-throughput screening (HTS) assays to identify agents that interact with or inhibit biological activity (i.e., affect enzymatic activity, binding activity, etc.) of a eukaryotic membrane protein polypeptide. HTS assays permit screening of large numbers of compounds in an efficient manner. In one embodiment, the eukaryotic membrane protein is an eukaryotic ion channel.

Cell-based HTS systems are contemplated to investigate the channel's receptor-ligand interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the channel polypeptide, i.e., rational design. One of skill in the art can, for example, measure the activity of the ion channel polypeptide using electrophysiological methods. Where the activity of the sample containing the agent is higher than the activity in the sample lacking the test compound, the agent has increased activity and comprises an agonist of the ion channel tested. Alternatively, where the activity of the sample containing the agent is lower than the activity in the sample lacking the agent, the agent has inhibited activity and comprises an antagonist of the ion channel tested.

The activity of the ion channels of the invention can also be determined by, as non-limiting examples, the ability to bind or be activated by certain ligands, including, but not limited to, known neurotransmitters, agonists and antagonists, including but not limited to serotonin, acetylcholine, nicotine, glutamate, and GABA.

Alternatively, the activity of the membrane protein (e.g., ion channel, gap junction, ionotropic receptor) can be assayed by examining activity such as ability to bind or be affected by sodium and calcium ions, hormones, chemokines, neuropeptides, neurotransmitters, nucleotides, lipids, odorants, and photons. In various embodiments of the method, the assay may take the form of an ion flux assay, a membrane potential assay, a yeast growth assay, a cAMP assay, an inositol triphosphate assay, a diacylglycerol assay, an Aequorin assay, a Luciferase assay, a FLIPR assay for [Ca²⁺]ᵢ, a mitogenesis assay, a MAP Kinase activity assay, an arachidonic acid release assay (e.g., using [³H]-arachidonic acid), and an assay for extracellular acidification rates, as well as other binding or function-based assays of activity that are generally known in the art.

Another potentially useful assay to examine the activity of a protein that mediates membrane flux (e.g., membrane protein) is electrophysiology (e.g., patch clamp, patch clamp arrays, or contractile behavior), the measurement of ion permeability across the cell membrane. This technique is described in, for example, Electrophysiology, A Practical Approach, D. I. Wallis editor, IRL Press at Oxford University Press, (1993), and Voltage and Patch Clamping with Microelectrodes, Smith et al., eds., Waverly Press, Inc for the American Physiology Society (1985).

Another assay to examine the activity of membrane proteins (e.g., ion channel, gap junction, ionotropic receptor) is through the use of the FLIPR Fluorometric Imaging Plate Reader system, developed by Dr. Vince Groppi of the Pharmacia Corporation to perform cell-based, HTS assays measuring, for example, membrane potential. Changes in plasma membrane potential correlate with the modulation of ion channels as ions move into or out of the cell. The FLIPR system measures such changes in membrane potential or intracellular calcium concentration. This is accomplished by loading cells expressing a gene encoding a protein that mediates ion flux with a cell-membrane permeant fluorescent indicator dye suitable for measuring changes in membrane potential such as diBAC (bis-(1,3-dibutylbarbituric acid) pentamethine oxonol, Molecular Probes). Alternatively, cell impermeant dyes can be introduced by electroporation. Thus the modulation of a protein that mediates ion flux, such as an ion channel, can be assessed with FLIPR and detected as changes in the emission spectrum of the diBAC or Fluo-4 dyes.

The present invention is particularly useful for screening compounds by using the channel in any of a variety of drug screening techniques. The compounds to be screened include (which may include compounds which are suspected to modulate the channel activity), but are not limited to, extracellular, intracellular, biologic or chemical origin. One skilled in the art can, for example, measure the formation of complexes between an ion and the compound being tested.

Alternatively, one skilled in the art can examine the diminution in complex formation between the channel and its substrate or other interacting molecules caused by the compound being tested. The activity of the channel polypeptides of the invention can be determined by, for example, examining the ability to bind or be activated by chemically synthesized peptide ligands. Alternatively, the activity of the channel polypeptides can be assayed by examining their ability to bind calcium ions, hormones, chemokines, neuropeptides, neurotransmitters, nucleotides, lipids, odorants, and photons. Alternatively, the activity of the channel polypeptides can be determined by examining the activity of effector molecules including, but not limited to, adenylate cyclase, phospholipases and ion channels. Thus, agents that can modulate the channel activity may alter ion channel function, such as a binding property of a channel or an activity such as ion selectivity.

In certain embodiments of the invention, methods of screening for compounds that modulate the channel activity comprise contacting test compounds with the channel and assaying for the presence of a complex between the compound and an ion. In such assays, the ligand is typically labeled. After suitable incubation, free ligand is separated from that present in bound form, and the amount of free or uncomplexed label indicates the ability of the particular compound to bind to the ion. Examples of such biological responses include, but are not limited to, the following: the ability to survive in the absence of a limiting nutrient in specifically engineered cells; changes in intracellular [Ca²⁺] as measured by fluorescent dyes (Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996,1,75-80). Binding of radiolabelled channel ligands can also be used to identify agents that interact directly with the channel. Assays are also available for the measurement of common second messengers, including but not limited to cAMP, phosphoinositides and arachidonic acid.

Agents that modulate activity or expression (e.g., agents that modulate the activity of a protein that mediates ion flux) may be identified by incubating a putative agent (or test compound) with a prokaryotic cell or with a unicellular protist transformed with a nucleic acid construct or composition of the invention and determining the effect of the putative agent on the channel activity or expression. The selectivity of a compound that modulates the activity of the channel can be evaluated by comparing its effects on the channel to its effect on other ion channel-modulating agents and to its effect on other ion channels (cross-screening). Selective ion channel-modulating agents may include, for example, antibodies and other proteins, peptides, or organic molecules that specifically bind to the ion channel polypeptide or an ion channel-encoding nucleic acid. Agents that modulate the channel activity will be therapeutically useful in treatment of diseases, injuries, and physiological conditions in which normal or aberrant membrane protein activity is involved. For example, agents that modulate the activity of an ion-channel will be therapeutically useful in the treatment of diseases, injuries, and physiological conditions in which ion channel activity is involved. Putative agents identified as modulating channel activity may be further tested in other assays including, but not limited to, in vivo models, in order to confirm or quantitate their activity. The channel polynucleotides and polypeptides, as well as the channel-modulating agents, may also be used in diagnostic assays for numerous diseases or conditions, including metabolic and cardiovascular diseases, inflammatory diseases, hormonal disorders, reproductive disorders, respiratory disorders, muscular skeletal disorders, and various neurological disorders.

The invention thus contemplates methods of screening for and identifying, characterizing, and/or optimizing agents that can modulate one or more activities of a membrane protein. By way of illustrative example, the invention contemplates methods of screening for and identifying, characterizing, and/or optimizing agents that modulate the activity of one or more ion channels. The modulation may be ion channel type-specific, for example, an agent can modulate activities of various calcium channels but does not modulate (or modulates to a significantly less extent) the activities of sodium channel, potassium channel, or chloride channel. Without being bound to any particular theory, such a type-selective agent may interact with Ca²⁺ or a calcium channel-specific domain of the ion channel polynucleotide or polypeptide with a high specificity. Alternatively, the modulation may be specific to each subtype or isoform of ion channels. For example, an agent may be a highly selective modulator of CatSper, but not other calcium channels; an agent may be a highly selective modulator of inward rectifier potassium channels, but not outward rectifier potassium channels. As will be appreciated by one of ordinary skill in the art, all of these modulator agents having different levels of specificity may have uses for different purposes, e.g., studying mechanisms underlying ion selectivity or specific function or biological activity of a particular subtype of an ion channel.

D. Agents

The present invention contemplates agents that function as modulators of the activity of a membrane protein (e.g., a protein that mediates membrane flux), thereby modulating ion flux. By “agents” or “candidate agents” herein is meant to include nucleic acids, peptides, polypeptides, peptidomimetics, small organic molecules, inorganic molecules, antisense oligonucleotides, RNAi constructs, antibodies, and ribozymes that function as antagonistic or agonistic candidate agents.

An agent of the invention may comprise an agonist of a membrane protein or, alternatively, an antagonist of a membrane protein. For example, an agent of the invention may comprise an agonist of an ion channel or an agent may comprise an antagonist of an ion channel.

The term “agonist,” as used herein, is meant to refer to an agent that mimics or upregulates (e.g., potentiates or supplements) bioactivity of the protein of interest. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide of interest with another molecule, e.g., a target molecule, a target peptide, or nucleic acid.

By “antagonist” herein is meant an agent that downregulates (e.g. suppresses or inhibits) bioactivity of the protein of interest. An antagonist can be a compound which inhibits or decreases the interaction between a protein and another molecule. For example, an antagonist of the invention may compete for binding to an ion channel against the ion channel's naturally occurring ligands or agonists. An antagonist can also be a compound that down-regulates expression of the gene of interest or which reduces the amount of the wild type protein present.

Agents that stimulate the channel's activity are useful as agonists in disease states or conditions characterized by insufficient channel signaling (e.g., as a result of insufficient activity of the channel ligand). Agents that block ligand-mediated channel signaling are useful as the channel antagonists to treat disease states or conditions characterized by excessive channel signaling. In addition, the ion channel-modulating agents in general, as well as channel polynucleotides and polypeptides, are useful in diagnostic assays for such diseases or conditions.

The agents of the invention exhibit a variety of chemical structures, which can be generally grouped into non-peptide mimetics of natural ion channel ligands, peptide and non-peptide allosteric effectors of ion channels, and peptides that may function as activators or inhibitors (competitive, uncompetitive and non-competitive) (e.g., antibody products) of a membrane protein. The invention does not restrict the sources for suitable agents, which may be obtained from natural sources such as plant, animal or mineral extracts, or non-natural sources such as small molecule libraries, including the products of combinatorial chemical approaches to library construction, and peptide libraries. Examples of organic modulating agents of ionotropic receptor membrane proteins are GABA, serotonin, acetylcholine, nicotine, glutamate, glycine, NMDA, and kainic acid. Examples of inorganic modulating agents that modulate activity of membrane proteins (e.g, ion channels) include ruthenium red.

Other assays can be used to examine enzymatic activity including, but not limited to, photometric, radiometric, HPLC, electrochemical, and the like, which are described in, for example, Enzyme Assays: A Practical Approach (R. Eisenthal and M. J. Danson, 1992, Oxford University Press).

Candidate agents contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides or organic molecules.

Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry.

Natural product libraries are collections of microorganisms, animals, plants, or marine organisms that are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282: 63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are-non-peptide combinatorial libraries. Still other libraries of interest include peptide, protein, peptidomimetic, multiparallel synthetic collection, recombinatorial, polypeptide, antibody, and RNAi libraries. For a review of combinatorial chemistry and libraries created therefrom, see Myers, Curr. Opin. Biotechnol. 8: 701-707 (1997). Identification of modulators through use of the various libraries described herein permits modification of the candidate “hit” or “lead” to optimize the capacity of the “hit” to modulate activity.

Numerous mechanisms exist to modulate the expression and/or activity of a particular mRNA or protein. Without being bound by theory, the present invention contemplates any of a number of methods for modulating the expression and/or activity of a particular ion channel. Furthermore, the invention contemplates any of a number of methods for modulating the expression and/or activity of a particular eukaryotic ion channel. Still furthermore, the invention contemplates combinatorial methods comprising either (i) the use of two or more candidate agents that modulate the expression and/or activity of a particular mRNA or protein, or (ii) the use of one or more candidate agents that modulate the expression and/or activity of a particular ion channel plus the use of one or more candidate agents that modulate the expression and/or activity of a second ion channel. Although certain embodiments use prokaryotic cells expressing a eukaryotic ion channel to screening these agents, an identified agent that can modulate the eukaryotic ion channel of interest will be further formulated to become a suitable agent for modulating the eukaryotic ion channel in eukaryotic cells, or more specifically, mammalian cells. It is further desirable to formulate the identified agent such that it will be suitable for modulating the eukaryotic ion channel in its native form in vivo.

The following are illustrative examples of methods for modulating the expression and/or activity of an mRNA or protein of an ion channel. These examples are in no way meant to be limiting, and one of skill in the art can readily select from among known methods of modulating expression and/or activity.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein and therefore may generally comprise antagonistic agents of the present invention, if they target a eukaryotic ion channel. Alternatively, antisense molecules may comprise agonistic agents of the present invention, if they target an antagonistic mRNA or protein modulator of the eukaryotic ion channel of choice.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO88/09810) or the blood-brain barrier (see, e.g., PCT Publication No. WO89/10134), hybridization-triggered cleavage agents (see, e.g., Krol et al., 1988, BioTechniques 6: 958-976) or intercalating agents (see, e.g., Zon, 1988, Pharm. Res. 5: 539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93: 14670 and in Eglom et al. (1993) Nature 365: 566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an anomeric oligonucleotide. An anomeric oligonucleotide forms specific double-stranded hybrids with complementary RNA in which, contrary to the usual units, the strands run parallel to each other (Gautier et al., 1987, Nucl. Acids Res. 15: 6625-6641). The oligonucleotide is a 2′-O-methylribonucleotide (Inoue et al., 1987, Nucl. Acids Res. 15: 6131-6148), or a chimeric RNA-DNA analogue (Inoue et al., 1987, FEBS Lett. 215: 327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site or the cells, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in prokaryotic cells (e.g., in an expression system or screening assay of the invention) or eukaryotic cells (e.g., in a pharmaceutical preparation of the invention). Such promoters can be inducible or constitutive. Such promoters include but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al, 1982, Nature 296: 39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo. As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell (e.g., a prokaryotic cell in a screening assay of the invention, or a eukaryotic cell or mammalian cell in a pharmaceutical preparation of the invention). In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiological conditions of the cell to the nucleotide sequence of at least a portion of the mRNA transcript for the gene to be inhibited (i.e., the “target” gene), e.g., a eukaryotic ion channel or a protein modulator of the eukaryotic ion channel. The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, for example, Heidenreich et al. (1997) Nucl. Acids Res, 25: 776-780; Wilson et al. (1994) J. Mol. Recog. 7: 89-98; Chen et al. (1995) Nucl. Acids Res 23: 2661-2668; Hirschbein et al. (1997) Antisense Nucl. Acid Drug Dev 7: 55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition. In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred. In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above.

Ribozymes molecules designed to catalytically cleave an mRNA transcripts can also be used to prevent translation of mRNA (see, e.g., PCT International Publication WO90/11364, published Oct. 4, 1990; Sarver et al., 1990, Science 247: 1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334: 585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224: 574-578; Zaug and Cech, 1986, Science, 231: 470-475; Zaug, et al., 1986, Nature, 324: 429-433; WO88/04300; Been and Cech, 1986, Cell, 47: 207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

Antibodies can be used as modulators of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of an ion channel by sterically hindering the proper ion channel subunits interactions or proper ion channel interactions with other molecules, or occupying active sites. Alternatively the binding of the antibody to an epitope on the particular protein may alter the conformation of that protein such that it is no longer able to properly function.

Monoclonal or polyclonal antibodies can be made using standard protocols (see, e.g., Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

In the context of the present invention, antibodies can be screened and tested to identify those antibodies that can modulate the function of a particular ion channel. One of skill in the art will recognize that not every antibody that is specifically immunoreactive with a particular channel will affect the function of that channel. However, one of skill in the art can readily test antibodies to identify those that are capable of stimulating or blocking the function of a particular ion channel. Alternatively, screening assays of the invention may be used to test antibodies against a particular eukaryotic ion channel.

The term antibody as used herein is intended to include fragments thereof which are also specifically reactive with a particular ion channel. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular ion channel in a biological process, or in a therapeutic context.

Variants polypeptides and peptide fragments can agonize or antagonize the function of a particular protein. Examples of such variants and fragments include constitutively active or dominant negative mutants of a particular protein. Agonistic or antagonistic variants may function in any of a number of ways, for example, as described herein. One of skill in the art can readily make variants comprising an amino acid sequence at least 60%, 70%, 75%, 80%, 85%, 90%, 95%, 98% or 99% identical to a particular ion channel, or a fragment thereof, and identify variants that agonize or antagonize the function of the wild type channel protein.

Similarly, one can make peptide mimetics that agonize or antagonize the function of a particular protein. Methods of making various peptide mimetics are well known in the art, and one of skill can readily make a peptide mimetic of a particular ion channel, or fragment thereof, and identify mimetics that agonize or antagonize the function of the wild type channel protein.

Small organic molecules can agonize or antagonize the function of a particular protein. By small organic molecule is meant a carbon contain molecule having a molecular weight less than 2500 amu, more preferably less than 1500 amu, and even more preferably less than 750 amu.

Small organic molecules can be readily identified by screening libraries of organic molecules and/or chemical compounds to identify those compounds that have a desired function. Without being bound by theory, small organic molecules may exert their agonistic or inhibitory function in any of a number of ways. For example, the small molecule may promote or compete against an ion “binding” (or entry into the pore) to its channel. If a transporter is involved, the agent may compete for the ion binding site on that transporter. Similarly, the small organic molecule may bind to and alter the confirmation of the channel protein, and thus agonize or antagonize the function of that channel. In another example, the small organic molecules may bind to another site on the channel and potentiate or disrupt an interaction required for the functionality of the channel. To illustrate, an protein may require a protein, vitamin, metal, or other cofactor for functionality, and the small organic molecule may disrupt this interaction. For example, for a ligand-gated ion channel, a small molecule agent may potentiate or disrupt the ligand's action upon the ion channel.

In addition to screening readily available libraries to identify small organic molecules with a particular modulating function (agonistic or antagonistic), the present invention contemplates the rational design and testing of small organic molecules that can modulate the function of a particular ion channel. For example, based on molecular modeling of the binding site of an ion channel, one of skill in the art can design small molecules that can occupy that binding pocket. Such small organic molecules would be candidate ion channel-modulating agents.

In addition to small organic molecules, agents within the scope of the present invention include inorganic molecules. Inorganic molecules can be identified from amongst libraries of inorganic molecules or by selectively screening individual or pools of candidate agents, and can agonize or antagonize the activity of a membrane protein.

As will be readily apparent in light of the detailed description of exemplary agonistic and antagonistic agents described herein, the invention contemplates agents that modulate the activity of membrane proteins via any of a number of mechanisms. The screening methods described herein are not biased in favor of identifying agents that function through a particular mechanism, but are instead based on the identification of agents that alter (increase or decrease) the functional activity of the membrane protein with respect to membrane flux. Accordingly, the present invention contemplates that certain agents may be able to modulate more than one membrane protein. Agents that modulate the activity of more than one protein include agents that modulate the activity of two or more of a particular class of membrane protein (e.g., two or more ion channels or two or more gap junctions), as well as agents that modulate the activity of membrane proteins of different classes (e.g., one or more ion channel and one or more gap junction). Furthermore, agents that modulate the activity of more than one membrane protein may modulate the activity in the same direction (e.g., agonize all of the membrane proteins, antagonize all of the membrane proteins) or may modulate the activity in different directions (e.g., agonize one or more membrane protein and antagonize one or more membrane protein). Additionally, the invention contemplates that certain agents may be specific, and may be able to modulate the activity of only one membrane protein.

E. Pharmaceutical Preparations

Agents for use in the methods of the present invention, as well as agents identified, characterized, and/or optimized by the subject methods may be conveniently formulated for administration with a biologically acceptable medium, such as water, buffered saline, polyol (for example, glycerol, propylene glycol, liquid polyethylene glycol and the like) or suitable mixtures thereof. Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Mack Publishing Co., Easton, Pa., USA 1985). These vehicles include injectable “deposit formulations.”

Methods of introduction may also be provided by rechargeable or biodegradable devices. Various slow release polymeric devices have been developed and tested in vivo in recent years for the controlled delivery of agents, including proteinacious biopharmaceuticals. A variety of biocompatible polymers (including hydrogels), including both biodegradable and non-degradable polymers, can be used to form an implant for the sustained release of an agent at a particular target site. Delivery of agents to a desired site can be attained by vascular administration via liposomal or polymeric nano- or micro-particles; slow-release vehicles implanted at the site of injury or damage; osmotic pumps implanted to deliver at the site of injury or damage; injection of agents at the site of injury or damage directly or via catheters or controlled release devices; injection into the cerebro-spinal fluid; injection intrapericardially.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

One or more agents may be administered to humans and other animals by any suitable route of administration. With regard to administration of agents to the brain, it is known in the art that the delivery of agents to the brain may be complicated due to the blood brain barrier (BBB). Accordingly, the application contemplates that agents may be administered directly to the brain cavity. For example, agents can be administered intrathecally or intraventricularly. Administration may be, for example, by direct injection, by delivery via a catheter or osmotic pump, or by injection into the cerebrospinal fluid.

However, although the BBB may present an impediment to the delivery of agents to the brain, it is also recognized that many agents, including nucleic acids, polypeptides and small organic molecules, are able to cross the BBB following systemic delivery. Therefore, the current application contemplates that agents may be delivered either directly to the desired site in the CNS or PNS, or may be delivered systemically.

With regard to administration of agents to myocardial tissue, it is known that agents can be administered in a variety of ways including systemically; via catheter, stent, intraluminal device, or wire; and via direct injection to the pericardium.

Actual dosage levels of the one or more agents may be varied so as to obtain an amount of the active ingredient which is effective to achieve a response in an animal, for example, the target ion channel's activity is modulated (potentiated or decreased) to a desired level. The actual effective amount can be determined by one of skill in the art using routine experimentation and may vary by mode of administration. Further, the effective amount may vary according to a variety of factors include the size, age and gender of the individual being treated. Additionally the severity of the condition being treated, as well as the presence or absence of other components to the individual's treatment regimen will influence the actual dosage. The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. Such additional compounds may include factors known to influence the proliferation, differentiation or migration of the particular cell type being manipulated. These additional compounds may be administered sequentially to or simultaneously with the agents for use in the methods of the present invention.

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the CNS, for example, intrathecal, intraventricular, intraspinal, or intracerebrospinal administration; (2) local administration to the myocardium, for example, via a stent, wire, intraluminal device, catheter, or via intrapericardial administration; (3) oral administration, for example, drenches (aqueous or non-aqueous solutions or suspensions), tablets, boluses, powders, granules, pastes for application to the tongue; (4) parenteral administration, for example, by subcutaneous, intramuscular or intravenous injection as, for example, a sterile solution or suspension; (5) topical application, for example, as a cream, ointment or spray applied to the skin; or (6) opthalamic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, e.g., Berge et al. (1977) Pharmaceutical Salts, J. Pharm. Sci. 66:1-19).

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1% to about 99% of active ingredient, preferably from about 5% to about 70%, most preferably from about 10% to about 30%.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention. These are particularly useful for injury and degenerative disorders of the eye including retinal detachment and macular degeneration.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without reduction or lose of activity.

(a) Containers or Vessels

The reagents included in the kits can be supplied in containers of any sort such that the life of the different components are preserved, and are not adsorbed or altered by the materials of the container. For example, sealed glass ampoules may contain lyophilized RE, RDF or buffer that have been packaged under a neutral, non-reacting gas, such as nitrogen. Ampoules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audiotape, etc. Detailed instructions may not be physically associated with the kit; instead, a user may be directed to an internet web site specified by the manufacturer or distributor of the kit, or supplied as electronic mail, or which is located on a server which can be accessed by the user. Access to a server containing instructions may either be freely available, or may be protected (e.g., by password) such that only specific individuals may have access to said instructional materials.

The compositions of the invention may be delivered to the cells and tissues, including the interstitial space of tissues, of the animal body, including those of skeletal muscle, cardiac muscle, skin, brain, lung, liver, spleen, bone marrow, thymus, heart, lymph, blood, bone, cartilage, pancreas, kidney, gallbladder, stomach, intestine, testis, vas deferens, ureter, urethra, epididymas, Fallopian tube, cervix, penis, vagina, ovary, uterus, rectum, nervous system, eye, gland, and connective tissue. Interstitial space of the tissues comprises the intercellular, fluid, mucopolysaccharide matrix among the reticular fibers of organs and tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. They may be conveniently delivered by injection into the tissues comprising these cells. Any apparatus known to the skilled artisan in the medical arts may be used to deliver the compositions of the invention to the desired site. These include, but are not limited to, syringes, stents, wires, intraluminal devices, and catheters.

In the case of damaged tissue throughout a subject, or in the blood vessels (or lymph system) themselves, then delivery into the circulation system may be desired. Any apparatus known to the skilled artisan in the medical arts may be used to deliver the compositions of the invention to the circulation system. These include, but are not limited to, syringes, stents, wires, intraluminal devices, and catheters. One convenient method is delivery via intravenous drip. Another approach would comprise implants, such as transdermal patches, stents, wires, intraluminal devices, and catheters that deliver the compositions of the invention over prolonged periods of time. Such implants may or may not be absorbed by the subject overtime.

During surgical procedures, the methods and compositions of the invention can be advantageously used to simplify the surgery of interest, such as reducing the amount of intervention, as well as to repair the damage wrought by the surgical procedure. The compositions of the invention may be delivered in a way that is appropriate for the surgery, including by bathing the area under surgery, implantable drug delivery systems, and matrices (absorbed by the body over time) impregnated with the compositions of the invention.

In the case of injuries to, or damaged tissues on, the exterior surfaces of a subject, direct application of the compositions of the invention is preferred. For example, a gauze impregnated with a compositions, may be directly applied to the site of damage, and may be held in place, such as by a bandage or other wrapping. Alternatively, the compositions of the invention may be applied in salves, creams, or other pharmaceutical compositions known in the art and meant for topical application.

F. Business Methods

The present application further contemplates methods of conducting businesses based on the compositions and methods of the invention. The discovery that an eukaryotic ion channel is well expressed in a prokaryotic cell provides for the first time increased capability to identify, characterize, and optimize known or new agents that can modulate activities of eukaryotic membrane proteins (e.g., proteins that mediate membrane flux) and further to provide methods and compositions using these membrane protein-modulating agents to treat a large number of injuries, diseases, and disorders related to misregulation of membrane protein activities. Exemplary membrane proteins that can be screened by the methods of the present invention include ion channels, gap junctions, and ionotropic receptors. The following example of the business methods contemplated by the present invention are described based on screening for agents that modulate the activity of one or more ion channels. However, the invention contemplates that similar methodology can be used to screen for and develop agents that modulate the activity of one or more gap junctions, ionotropic receptors, transporters, or the like.

In one aspect, the invention provides a method of conducting a drug discovery business comprising: identifying, characterizing, and/or optimizing one or more agents that modulate activity of a eukaryotic ion channel according to the screening methods described above; conducting therapeutic profiling of the one or more agents identified by the screening methods for efficacy and toxicity in one or more animal models; and formulating a pharmaceutical preparation comprising one or more agents identified by the therapeutic profiling as having an acceptable therapeutic profile. Certain embodiments further comprise establishing a system for distributing the pharmaceutical preparation for sale and/or establishing a sales group for marketing the pharmaceutical preparation.

In another aspect, the invention provides a method of conducting a gene therapy business comprising: examining patients with an injury or disease that relates to ion channel function or activities; administering to said patient an amount of an agent effective to treat the injury or disease; and monitoring the patients during and after treatment to assess efficacy of the treatment. The method of conducting a gene therapy business may optionally include a method of billing a patient or the patient's insurance carrier. Furthermore, the method includes the use of agents comprising nucleic acids, for example, nucleic acids comprising expression vectors.

In another aspect, the present invention provides a method of conducting a drug discovery business comprising: identifying, by the subject assays, one or more agents which modulate ion channel activities; determining if an agent identified in such an assay, or an analog of such an agent, modulates an ion channel in vivo and/or in vitro; conducting therapeutic profiling of an agent so identified for efficacy and toxicity in one or more animal models; and formulating a pharmaceutical preparation including one or more agents identified as having an acceptable therapeutic profile and which modulate ion channel activities.

In one embodiment, the drug discovery business further includes the step of establishing a system for distributing the pharmaceutical preparation for sale, and may optionally include establishing a sales group for marketing the pharmaceutical preparation.

In certain embodiments, the initially identified agents can be subjected to further lead optimization, e.g., to further refine the structure of a lead compound so that potency and activity are maintained but balanced with important pharmacological characteristics including: solubility, permeability, bioavailability, toxicity, mutagenicity, pharmacokinetics (absorption, distribution, metabolism, elimination of the drug).

Structural modifications are made to a lead compound to address issues with the parameters listed above. These modifications however, must take into account possible effects on the molecule's potency and activity. For example, if the solubility of a lead compound is poor, changes can be made to the molecule in an effort to improve solubility; these modifications, however, may negatively affect the molecule's potency and activity. SAR data are then used to determine the effect of the change upon potency and activity. Using an iterative process of structural modifications and SAR data, a balance is created between these pharmacological parameters and the potency and activity of the compound.

Candidate agents, or combinations thereof, must them be tested for efficacy and toxicity in animal models. Such therapeutic profiling is commonly employed in the pharmaceutical arts. Before testing an experimental drug in humans, extensive therapeutic profiling (preclinical testing) must be completed to establish initial parameters for safety and efficacy. Preclinical testing establishes a mechanism of action for the drug, its bioavailability, absorption, distribution, metabolism, and elimination through studies performed in vitro (that is, in test tubes, beakers, petri dishes, etc.) and in animals. Animal studies are used to assess whether the drug will provide the desired results. Varying doses of the experimental drug are administered to test the drug's efficacy, identify harmful side-effects that may occur, and evaluate toxicity.

Briefly, one of skill in the art will recognize that the identification of a candidate agent which modulates activities of a target ion channel in a drug based screen is a first step in developing a pharmaceutical preparation useful for modulating ion channel activities either in vitro or in vivo. Administration of an amount of said pharmaceutical preparation effective to modulate the ion channel to a desired level must be both safe and effective. Early stage drug trials, routinely used in the art, help to address concerns of the safety and efficacy of a potential pharmaceutical. In the specific case of an ion channel-modulating agent, efficacy of the pharmaceutical preparation could be readily evaluated in normal or transformed cell lines, or in vivo or in vitro in a mouse or rat model. Briefly, mice or rats could be administered varying doses of said pharmaceutical preparations over various time schedules. The route of administration would be appropriately selected based on the particular characteristics of the agent and on the cell type in which modulation of activities of an ion channel or ion channels to a certain level is desired. Control mice can be administered a placebo (e.g., carrier or excipient alone).

In one embodiment, the step of therapeutic profiling includes toxicity testing of compounds in cell cultures and in animals; analysis of pharmacokinetics and metabolism of the candidate drug; and determination of efficacy in animal models of diseases. In certain instances, the method can include analyzing structure-activity relationship and optimizing lead structures based on efficacy, safety and pharmacokinetic profiles. The goal of such steps is the selection of drug candidates for pre-clinical studies to lead to filing of Investigational New Drug applications (“IND”) with the Food and Drug Administration (“FDA”) prior to human clinical trials.

Between lead optimization and therapeutic profiling, one goal of the subject method is to develop an ion channel-modulating agent which has minimal side-effects. In the case of agents for in vitro use, the lead compounds should not be exceptionally toxic to cells in culture, should not be mutagenic to cells in culture, and should not be carcinogenic to cells in culture. In the case of agents for in vivo use, lead compounds should not be exceptionally toxic (e.g., should have only tolerable side-effects when administered to patients), should not be mutagenic, and should not be carcinogenic.

By “toxicity profiling” is meant the evaluation of potentially harmful side-effects which may occur when an effective amount of a pharmaceutical preparation is administered. A side-effect may or may not be harmful, and the determination of whether a side effect associated with a pharmaceutical preparation is an acceptable side effect is made by the FDA during the regulatory approval process. This determination does not follow hard and fast rules, and that which is considered an acceptable side effect varies due to factors including: (a) the severity of the condition being treated, and (b) the availability of other treatments and the side-effects currently associated with these available treatments. For example, chemotheraputic drugs are an important part of the standard therapy for many forms of cancer. Although chemotherapeutics themselves can have serious side-effects including hair-loss, severe nausea, weight-loss, and sterility, such side-effects are considered acceptable given the severity of the disease they aim to treat.

In the context of the present invention, whether a side-effect is considered significant will depend on the condition to be treated and the availability of other methods to treat that condition. For example, the ion channel-modulating agent may be used to treat Long QT syndrome resulting from structural abnormalities in the potassium channels of the heart, which predispose affected persons to an accelerated heart rhythm (arrhythmia). This can lead to sudden loss of consciousness and may cause sudden cardiac death in teenagers and young adults who are faced with stress factors ranging from exercise to loud sounds. However, the level of impairment in the health of individuals with tissue damages due to the syndrome varies greatly depending on the overall health of the individual and the extent of damage. These factors must be considered in assessing whether a side-effect is reasonable. By way of another example, the ion channel-modulating agent may be formulated to treat Cystic Fibrosis by modulating CFTR (a Cl⁻ channel) activities. Cystic fibrosis is a genetic disease affecting approximately 30,000 children and adults in the United States. A defective gene causes the body to produce an abnormally thick, sticky mucus that clogs the lungs and leads to life-threatening lung infections. In this case, the extent to which a side-effect is considered acceptable may be weighed differently at different stages of this condition given that, at the stage of lung-infections, the condition is likely life-threatening.

By way of another example, the agent may modulate the activity of a CatSper ion channel, and such agent may be useful in methods of regulating fertility. One preferred class of CatSper modulating agents could be developed and sold as a contraceptive. Another preferred class of CatSper modulating agents could be developed and sold as a fertility treatment, or as part of a fertility treatment plan.

Toxicity tests can be conducted in tandem with efficacy tests, and mice administered effective doses of the pharmaceutical preparation can be monitored for adverse reactions to the preparation.

An agent or agents which modulate ion channel activities, and which are proven safe and effective in animal studies, can be formulated into a pharmaceutical preparation. Such pharmaceutical preparation can then be marketed, distributed, and sold. Sale of these agents may either be alone, or as part of a therapeutic regimen including evaluation by a physician, appropriate treatment, and appropriate after-care in coordination with the treating physician or with another licensed physician or health care provider.

The above examples are meant to be purely illustrative, and the present invention similarly contemplates a drug discovery business based on screening to identify modulators of other membrane proteins. In one embodiment, the drug discovery business is based on screening to identify and further develop agents that modulate the activity of a gap junction. Given the role of gap junctions in, for example, the nervous system, such agents can be used to develop effective treatments for injuries and diseases of the central and peripheral nervous system.

In another embodiment, the drug discovery business is based on screening to identify and further develop agents that modulate the activity of an ionotropic receptor. Given the role of ionotropic receptor in, for example, the nervous system, such agents can be used to develop effective treatments for injuries and diseases of the central and peripheral nervous system.

In another embodiment, the drug discovery business is based on screening to identify and further develop agents that modulate the activity of a transporter. Given the role of transporters in, for example, the nervous system, such agents can be used to develop effective treatments for injuries and diseases of the central and peripheral nervous system.

The references cited throughout the application are hereby expressly incorporated in full by reference.

### Exemplification

The invention now being generally described, it will be more readily understood by reference to the following examples, which are included merely for purposes of illustration of certain aspects and embodiments of the present invention, and are not intended to limit the invention.

### Example 1

### Exemplary Prokaryotic Cells-Bacteria

The bacteria are a modified E. Coli strain from Novagen “Rosetta-Gami B (DE3) pLysS,” having the following characteristics:
1) BL21 derived (protease deficient);
2) Carrying an IPTG inducible T7 polymerase;
3) Lac permease mutation to allow concentration dependent induction by IPTG;
4) T7 lysozyme gene to degrade T7 polymerase and ensures tight regulation of expressed proteins;
5) 6 rare tRNAs driven by native promoters;
6) TrxB (thioredoxin) to facilitate folding and disulfide bond formation;
7) Gor (Glutathione reductase) to facilitate disulfide bond formation in cytoplasm.

### Example 2

### Exemplary Protocol for Expressing and Detecting Activities of Eukaryotic Ion Channels in Bacteria

1) 0.5-2 mL of an overnight bacteria (already transformed with a nucleic acid construct or composition encoding a eukaryotic Calcium ion channel and an aequorin marker) culture is innoculated into 25 mL of LB-Amp;
2) Bacteria are grown to an OD (optical density) of ˜0.6;
3) 1 mM IPTG added for 2-hour induction;
4) Bacteria are spun down, resuspended in ½ volume of nominally divalent free ringers and loaded with coelenterazine;
5) Bacteria are rinsed and loaded into 96 well plate (90-95 mL/well);
6) Plate is put in a plate reader, e.g., a Wallac Victor 3;
7) Plate is shaken for 2 sec then rested for 5 seconds;
8) 10 reads are taken 1 second apart;
9) 5 μL of 20 mM CaCl₂ is injected;
10) 50 reads are taken 1 second apart;
Note: When a blocker is present, it is added before the plate is put in the reader.

### Example 3

### A Human Calcium Channel Modulates Calcium Ion Flux in Bacterial Cells

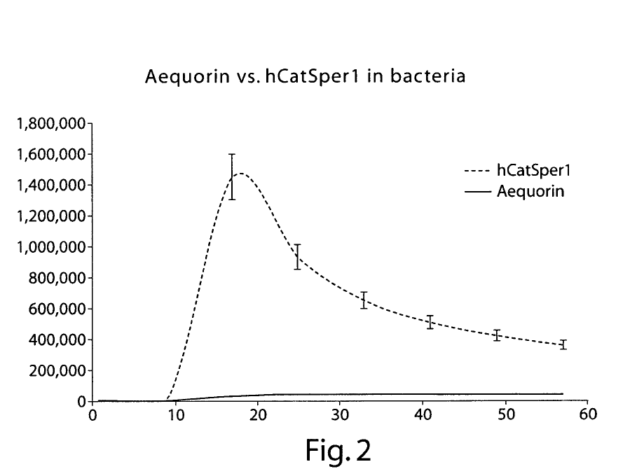

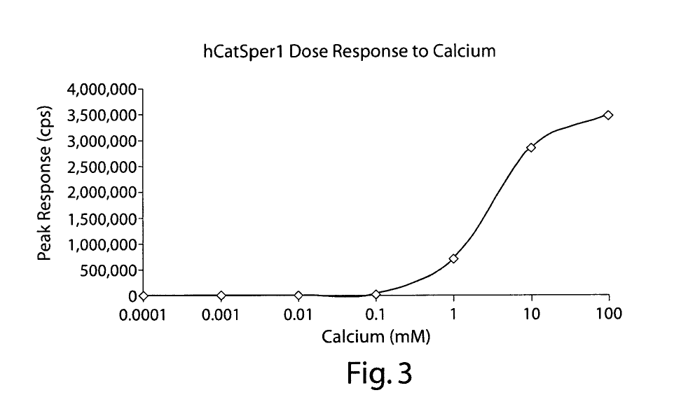

Human CatSper 1 and the detectable reporter aeuquorin were co-expressed in bacterial cells using the expression plasmids depicted in FIGS. 1 and 6. The calcium flux modulating activity of CatSper1 was compared to that of bacterial cells expressing aeuquorin alone. FIGS. 2 and 3 summarize experiments performed following the protocol outlined in Example 2. These results indicated that the human calcium channel CatSper1 effectively modulates calcium flux in bacterial cells, as assayed using a detectable marker that can indicate changes in ion flux (e.g., aequorin).

### Example 4

### A Human Calcium Channel Specifically Modulates Calcium Flux in Bacterial Cells

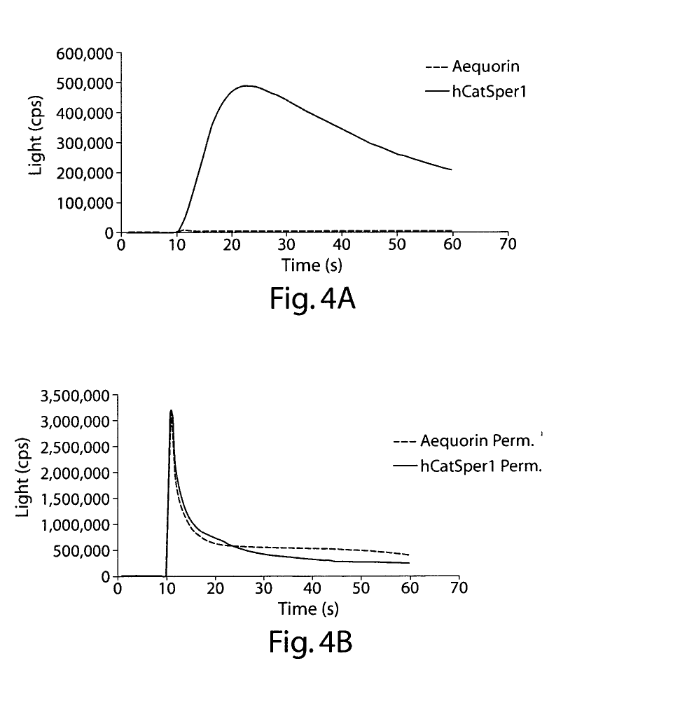

We conducted experiments to address the specificity of CatSper1 activity in bacterial cells, and the results of these experiments are summarized in FIG. 4. As outlined in detail above, calcium flux in bacterial cells expressing CatSper1+aequorin were compared to calcium flux in bacterial cells expressing aequorin alone. FIG. 4a shows that expression of a eukaryotic ion channel (hCatSper1) mediates calcium flux across a bacterial cell. This flux mediating activity of CatSper1 is abrogated if the bacterial cells are permeabilized. FIG. 4b shows that this regulation of calcium flux is specific and is disrupted when the cells are permeabilized with lysozyme and deoxycholate.

### Example 5

### Eukaryotic Ion Channels are Functional in Bacterial Cells

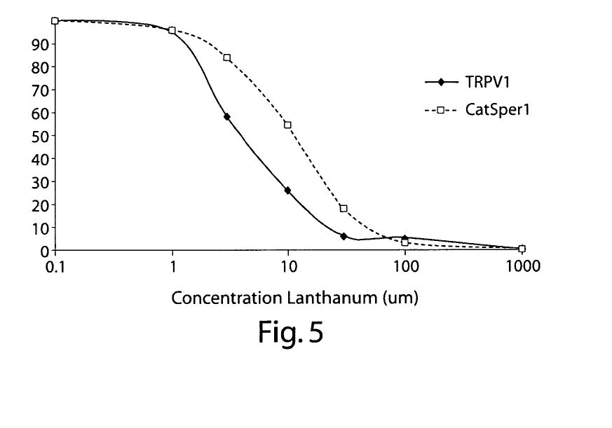

In addition to CatSper1, we have conducted additional experiments demonstrating that other eukaryotic ion channels can be expressed and are functional in bacterial cells. As outlined above, the functional activity of an ion channel can be demonstrated by showing that the expressed ion channel mediates ion flux in the bacterial cell. A further indicator of the functional activity of the ion channel is whether its activity is sensitive to treatment with agonistic or inhibitory agents. FIG. 5 summarizes experiments conducted to address this issue. Briefly, FIG. 5 demonstrates that both the human CatSper 1 calcium channel and the rat TrpV1 calcium channel mediate calcium ion flux when expressed in bacterial cells. Furthermore, the calcium-flux mediating activity of the channels is inhibited following addition of the calcium channel inhibitor, lanthanum.

### Example 6

### Eukaryotic Ion Channels Specifically Modulate Ion Flux in Bacterial Cells

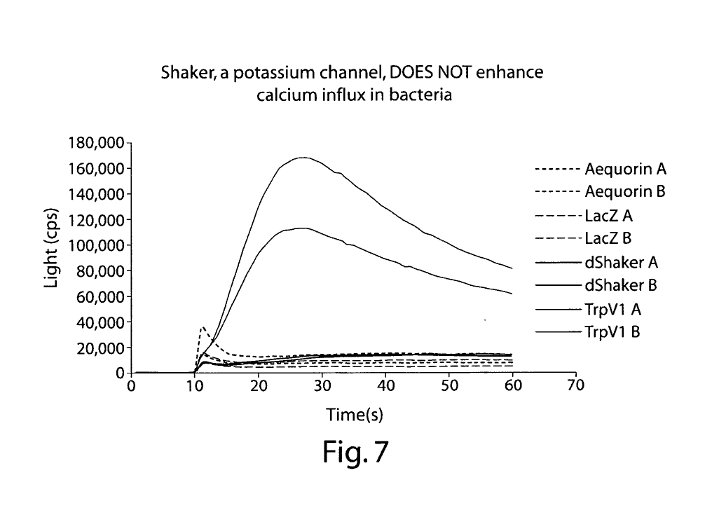

FIG. 7 shows that eukaryotic channels expressed in bacterial cells specifically mediate ion flux (e.g., mediate flux of the same ions that the channel normally acts to modulate in eukaryotic cells). Expression of the eukaryotic calcium channel TrpV1 mediated calcium influx in bacterial cells. However, the Drosophila potassium channel Shaker (dShaker), which normally functions to modulate potassium flux, did not mediate calcium flux when expressed in bacterial cells.

Briefly, E. Coli bacteria were produced that express either Aequorin (Aequorin), Aequorin+LacZ (LacZ), Aequorin+the drosophila potassium channel Shaker (Shaker) or Aequorin+the calcium channel TRPV1 (TRPV1). Two independent bacterial colonies for each condition were tested (A & B). The bacteria were grown and loaded with Coelenterazine in divalent free conditions. The bacteria were then loaded into a plate luminometer and imaged for 60 seconds. After 10 seconds calcium was added to the solution. Bacteria expressing the TRPV1 calcium channel, but not bacteria expressing the Shaker potassium channel, mediated calcium influx. Failure of the Shaker potassium channel to mediate calcium influx was not due to a lack of protein expression which was verified by Western blot with anti-shaker antibodies.

### Example 7

### Trp Channel Inhibitors

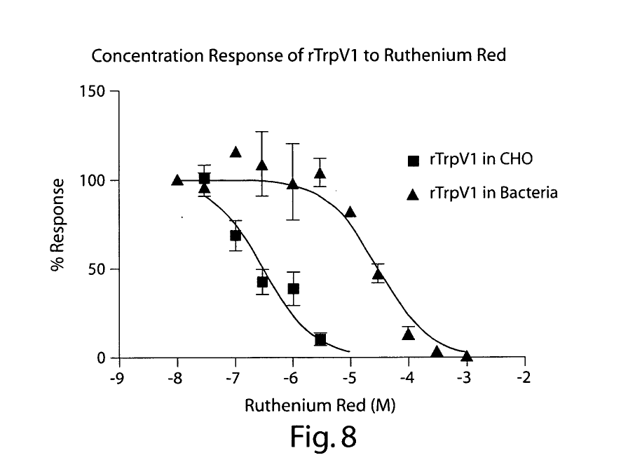

FIG. 8 shows that rat TrpV1 mediates calcium flux when expressed in either CHO cells or bacterial cells. The Trp channel blocker ruthenium red inhibits TrpV1 mediated calcium flux in bacterial cells or CHO cells. Although this particular non-specific Trp inhibitor is somewhat less potent in bacterial cells, it is active. Additionally, although the IC50 differs, the calculated Hill slope values are not significantly different between the CHO cells and the bacterial cells.

### Example 8

### Two Different Eukaryotic Calcium Channels

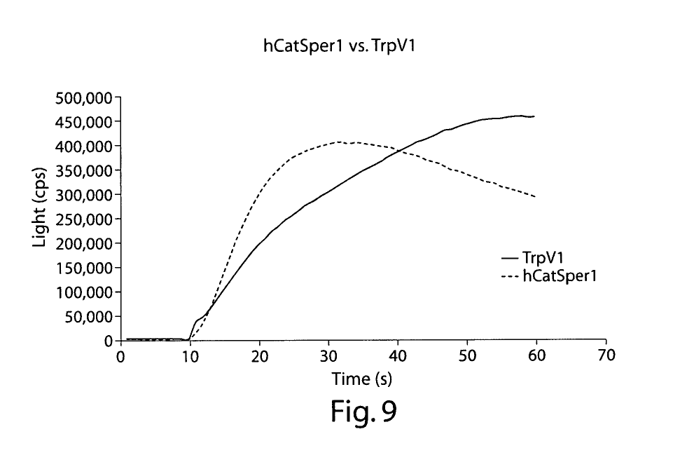

FIG. 9 shows that two different eukaryotic calcium channels, hCatSper1 and rTrpV1, can be efficiently expressed in bacterial cells to produce functional calcium channels that mediate calcium flux across the bacterial cell. Each ion channel was individually co-expressed with aequorin using the expression plasmid outlined in FIG. 6.

### REFERENCES

WO03/054141
WO04/015067
WO04/015066
WO03/040323
WO02/090567
Saez et al. (2003) Physiol Rev 83: 1359-1400.
Chang et al. (2003) Ear Hear. 24: 314-323.
Nikpoor et al. (2004) Human Reprod. 19: 124-128.
Carlson et al. (2003) PNAS 100: 14864-14868.
Lobley et al. (2003) Reprod. Biol. Endocrinol. 1: 53.
Quill et al. (2001) PNAS 98: 12527-12531.
Ren et al. (2001) Nature 413: 603-609.
Clapham (2003) Nature 426 : 517-524.
Clapham (2002) Science 295 : 2228-2229.
Runnels et al. (2002) Nature Cell Biology 4 :329-336.
Greka et al. (2003) Nat. Neuroscience 6: 837-845.
Yue et al. (2002) J. Gen. Physiol 120: 845-853.
Xu et al. (2002) Nature 418: 181-186.
Corey et al. (1998) Journal of Biol Chem 273: 27499-27504.
Corey et al. (1998) Journal of Biol Chem 273: 5271-5278.
Wickman et al. (2002) Gene 284: 241-250.
Jelacic et al. (2000) Journal of Biol Chem 275: 36211-36216.
Wickman et al. (2000) Journal of Neuroscience 20: 5608-5615.
Kennedy et al. (1999) Journal of Biol Chem 274: 2371-2382.

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein. Such equivalents are intended to be encompassed by the following claims.

TABLE 1
GenBank Accession No. for non-limiting examples of membrane
protein nucleic acid and amino acid sequences. The nucleic acid
and amino acid sequences provided with references to GenBank
accession numbers are hereby incorporated by reference in their
entirety. Furthermore, the literature references cited at each of
these GenBank accession numbers are hereby
incorporated by reference in their entirety.

| Polypeptide | GenBank Accession No. |
|---|---|
| Human CatSper1 | NM_053054 |
| Mouse CatSper1 | NM_139301 |
| Human CatSper2 | NM_054020; NM_172095; |
| | NM_172096; NM_172097 |
| Mouse CatSper2 | NM_153075 |
| Human CatSper3 | NM_178019 |
| Mouse CatSper3 | NM_029772 |
| Human CatSper4 | XM_371237 |
| Mouse CatSper4 | AY263400 |
| Human GIRK1 | NM_002239 |
| Mouse GIRK1 | NM_008426 |
| Human GIRK2 | NM_002240 |
| Mouse GIRK2 | NM_010606 |
| Human GIRK3 | NM_004983 |
| Mouse GIRK3 | NM_008429 |
| Rat GIRK3 | NM_053834 |
| Human GIRK4 | NM_000890 |
| Mouse GIRK4 | NM_010605 |
| Chick GIRK4 | U71060 |
| Mouse TRPM7 | NM_021450 |
| Human TRPM7 | NM_017672 |
| Human TRPM6 | AY333283; AY333284; AY333285 |
| Human TRPM8 | AY532375; AY532376; NM_024080 |
| Mouse TRPM8 | NM_134252 |
| Human TRPV1 | NM_080704; NM_080705; |
| | NM_080706; NM_018727 |
| Rat TRPV1 | NM_031982 |
| Mouse TRPV1 | NM_001001445 |
| Human TRPV2 | NM_016113 |
| Rat TRPV2 | NM_017207 |
| Mouse TRPV2 | NM_011706 |
| Human TRPV3 | NM_145068 |
| Mouse TRPV3 | NM_145099 |
| Human TRPV6 | NM_018646 |
| Rat TRPV6 | NM_053686 |
| Mouse TRPV6 | NM_022413 |
| Human TRPC2 | XR_000147 |
| Mouse TRPC2 | NM_011644 |
| Rat TRPC2 | NM_022638 |
| Human TRPC4 | NM_016179 |
| Cow TRPC4 | NM_174478 |
| Rat TRPC4 | NM_080396 |
| Mouse TRPC4 | NM_016984 |
| Human TRPC3 | NM_003305 |
| Human TRPC6 | NM_004621 |
| Mouse TRPC6 | NM_013838 |
| Human SLC6A9 | NM_006934 |
| Cow SLC6A9 | NM_174612 |
| Mouse SLC6A9 | NM_008135 |
| Human SLC6A3 | NM_001044 |
| Human SLC6A4 | NM_001045 |
| Human Dopamine | NM_000797 |
| receptor D4 | |
| Rat Cholinergic receptor | NM_080773 |
| Human Serotonin receptor | NM_006028; NM_000867; NM_000864 |
| Rat Serotonin receptor | NM_022225 |
| Mouse Serotonin receptor | NM_008313 |
| Human GABA receptor theta | NM_018558 |
| Human GABA receptor rho 1 | NM_002042 |
| Mouse GABA receptor rho 1 | AF024620 |
| Rat GABA receptor rho 1 | NM_017291 |
| Human GABA receptor rho 2 | NM_002043 |
| Mouse GABA receptor rho 2 | AF024621 |
| Rat GABA receptor rho 3 | NM_138897 |
| Mouse GABA gamma 2 | NM_177408; NM_008073 |
| Mouse GABA alpha 1 | NM_010250 |
| Human GABA alpha 3 | AF166361; AF166360; AF166359 |
| Human GABA beta 1 | NM_000812 |
| Rat GABA betal 1 | NM_031028 |
| Mouse GABA beta 2 | NM_008070 |
| HumanGABA beta 3 | NM_000814; NM_021912 |
| Human Connexin 30 | NM_006783 |
| Rat Connexin 30 | NM_053388 |
| Human Connexin 31 | NM_024009 |
| Rat Connexin 31 | M59936 |
| Xenopus Connexin 31 | AY057997 |
| Human Connexin 32 | NM_000166 |
| Chick Connexin 32 | AY363307 |
| Mouse Connexin 32 | M63802 |
| Human Connexin 26 | NM_004004 |
| Human Connexin 37 | NM_002060 |
| Human Connexin 40 | NM_005266; NM_181703 |
| Rat Connexin 40 | AF021806 |
| Human Connexin 43 | NM_000165 |
| Xenopus tropicalis Connexin | AY043270 |
| 43 | |
| Cow Connexin 43 | NM_174068 |
| Fish Connexin 43 | NM_131038 |
| Human Connexin 45 | NM_005497 |
| Xenopus Connexin 45 | BC042343 |
| Chick Connexin 45 | M35044 |
| Human Connexin 46 | NM_021954 |
| Rat Connexin 46 | NM_024376 |
| Human Connexin 50 | NM_005267 |
| Rat Connexin 50 | AB078344 |
| Human Connexin 59 | AF179597 |
| Human P2X1 | NM_002558 |
| Rat P2X1 | NM_012721 |
| Mouse P2X1 | NM_008771 |
| Human P2X2 | NM_174873; NM_174872 |
| Rat P2X2 | NM_053656 |
| Human P2X3 | NM_002559 |
| Rat P2X3 | NM_031075 |
| Mouse P2X3 | NM_145526 |
| Fish P2X3 | NM_131623 |
| Human P2X4 | NM_175567; NM_002560 |
| Rat P2X4 | NM_031594 |
| Xenopus P2X4 | AF308152; AF308153 |
| Human P2X5 | NM_175080; NM_002561 |
| Rat P2X5 | NM_080780 |
| Mouse P2X5 | NM_033321 |
| Human P2X7 | NM_177427; NM_002562 |
| Rat P2X7 | NM_019256 |
| Mouse P2X7 | NM_011027 |
| Xenopus P2X7 | AJ345114 |

1. A nucleic acid construct, comprising
(a) a first nucleic acid encoding a eukaryotic membrane protein that mediates ion flux and
(b) a second nucleic acid encoding a detectable marker,
wherein the first nucleic acid and the second nucleic acid are operably linked to a promoter, and wherein the detectable marker indicates a change in ion concentration mediated by the membrane protein.

2. The nucleic acid construct of claim 1, wherein the first nucleic acid and second nucleic acid are operably linked to one promoter.

3. The nucleic acid construct of claim 1, wherein the first nucleic acid is operably linked to a first promoter, and the second nucleic acid is operably linked to a second promoter.

4. The nucleic acid construct of claim 3, wherein the first promoter and the second promoter are different promoters.

5. The nucleic acid construct of claim 3, wherein the first promoter and the second promoter are the same promoters.

6. A nucleic acid composition comprising
(a) a first nucleic acid construct comprising a nucleic acid encoding a eukaryotic membrane protein that mediates ion flux and
(b) a second nucleic acid construct comprising a nucleic acid encoding a detectable marker, wherein
the detectable marker indicates a change in ion concentration mediated by the membrane protein.

7. The nucleic acid construct or nucleic acid composition of claim 1 or 6, wherein the eukaryotic membrane protein is a vertebrate or invertebrate ion channel.

8. The nucleic acid construct or nucleic acid composition of claim 7, wherein the eukaryotic membrane protein is a mammalian, plant, reptilian, amphibian, fish, or avian ion channel.

9. The nucleic acid construct or nucleic acid composition of claim 1, wherein the eukaryotic membrane protein is a chimeric ion channel or a mutant ion channel.

10. The nucleic acid construct or nucleic acid composition of claim 1, wherein the eukaryotic membrane protein is a eukaryotic ion channel selected from the group consisting of a calcium channel, a sodium channel, a potassium channel, a chloride channel, or a non-selective cation channel.

11. The nucleic acid construct or nucleic acid composition of claim 1, wherein the detectable marker comprises a luminescent, fluorescent, or calorimetric marker.

12. A prokaryotic cell transformed with and expressing a nucleic acid construct comprising a nucleic acid encoding a eukaryotic membrane protein.

13. The prokaryotic cell of claim 12, wherein the cell is a bacterial cell.

14. The prokaryotic cell of claim 12, further comprising a nucleic acid construct that comprises a nucleic acid encoding a detectable marker.

15. The prokaryotic cell of claim 12, further comprising a detectable marker.

16. The prokaryotic cell of claim 14, wherein the detectable marker is a luminescent marker, a fluorescent marker, or a colorometric marker.

17. The prokaryotic cell of claim 12, wherein the cell expresses two or more eukaryotic membrane proteins.

18. A unicellular protist transformed with and expressing a nucleic acid construct comprising a nucleic acid encoding a eukaryotic membrane protein.

19. The unicellular protist of claim 18, wherein the cell is a ciliated cell or a flagellated cell.

20. The unicellular protist of claim 18, further comprising a nucleic acid construct that comprises a nucleic acid encoding a detectable marker.

21. The unicellular protist of claim 18, further comprising a detectable marker.

22. The unicellular protist of claim 20, wherein the detectable marker is a luminescent marker, a fluorescent marker, or a colorometric marker.

23. A method of expressing a eukaryotic membrane protein in a prokaryotic cell comprising: transforming the prokaryotic cell with the nucleic acid construct of claim 1.

24. A method of identifying, characterizing, or optimizing agents that modulate activity of a eukaryotic membrane protein, comprising
(a) contacting a prokaryotic cell transformed with and expressing the nucleic acid construct or nucleic acid composition of claim 1 with one or more candidate agents, and
(b) detecting a change in the detectable marker in the presence of the one or more agents in comparison with the absence of the one or more agents,
wherein one or more candidate agents that cause a change in expression of the detectable marker are agents that modulate the activity of the membrane protein.

25. The method of claim 24, wherein the one or more agents that modulate the activity of the membrane protein antagonize the activity of the membrane protein.

26. The method of claim 24, wherein the one or more agents that modulate the activity of the membrane protein agonize the activity of the membrane protein.

27. The method of claim 24, wherein the eukaryotic membrane protein is a vertebrate or invertebrate ion channel

28. The method of claim 27, wherein the eukaryotic membrane protein is a mammalian, plant, avian, reptilian, fish, or amphibian ion channel.

29. The method of claim 24, wherein the detectable marker comprises a luminescent marker, a fluorescent marker, or a colorometric marker.

30. An agent identified by the method of claim 24, formulated in a pharmaceutically acceptable carrier.

31. Use of an agent identified by the method of claim 24 in the manufacture of a medicament for modulating the activity of a eukaryotic membrane protein.

32. A method of expressing a eukaryotic membrane protein in a unicellular protist comprising: transforming the unicellular protist with the nucleic acid construct of claim 1.

33. A method of identifying, characterizing, or optimizing agents that modulate activity of a eukaryotic membrane protein, comprising
(a) contacting a unicellular protist transformed with and expressing the nucleic acid construct or nucleic acid composition of claim 1 with one or more candidate agents, and
(b) detecting a change in the detectable marker in the presence of the one or more agents in comparison with the absence of the one or more agents,
wherein one or more candidate agents that cause a change in expression of the detectable marker are agents that modulate the activity of the membrane protein.

34. The method of claim 33, wherein the one or more agents that modulate the activity of the membrane protein antagonize the activity of the membrane protein.

35. The method of claim 33, wherein the one or more agents that modulate the activity of the membrane protein agonize the activity of the membrane protein.

36. The method of claim 33, wherein the eukaryotic membrane protein is a vertebrate or invertebrate ion channel

37. The method of claim 33, wherein the unicellular protist is a ciliated protist or a flagellated protist.

38. Use of an agent identified by the method of claim 33 in the manufacture of a medicament for modulating the activity of a eukaryotic membrane protein.

39. A prokaryotic cell transformed with and expressing the nucleic acid construct of claim 1.

40. The prokaryotic cell of claim 39, wherein the cell is a bacterial cell.
